(19) United States
(12) Patent Application Publication
(10) Pub. No.: US 2022/0114681 A1
(43) Pub. Date: Apr. 14, 2022

(54) Pollination Predictor System and Method

(71) Applicant: Accelerated Ag Technologies, LLC, Ankeny, IA (US)

(72) Inventors: Jason Cope, Ankeny, IA (US); Mark E. Westgate, Ames, IA (US); Todd Krone, Des Moines, IA (US)

(73) Assignee: Accelerated Ag Technologies, LLC, Ankeny, IA (US)

(21) Appl. No.: 17/500,644

(22) Filed: Oct. 13, 2021

Related U.S. Application Data
(60) Provisional application No. 63/091,433, filed on Oct. 14, 2020.

(51) Int. Cl. G06Q 50/02; A01G 22/05; G06F 30/20; A01H 1/02

(52) U.S. Cl. CPC G06Q 50/02; A01H 1/027; G06F 30/20; A01G 22/05

(57) ABSTRACT

Provided are methods for both pollinating and simulating pollination of a crop plant having one or more stigmas that are receptive to pollen and that produces at least one seed, grain, or fruit of interest. Methods of the present invention include ingesting input data, such as reproductive maturity data, for a population of the crop plant, sufficient to determine one or more days on which the crop plant will be receptive to pollen. The input data is modeled to generate the amount of receptive stigmas in the population, the effect of intentionally applying pollen during each time step to transform the number of receptive stigmas to a modeled output of seed, grain, or fruit of interest, and generating one or more time steps during which intentional pollination is modeled to provide a greater harvest of the seed, grain, or fruit of interest than other of said time steps. The crop may be intentionally pollinated during at least one of said time steps during which intentional pollination is modeled to provide a greater harvest of the seed, grain, or fruit of interest.

This application claims priority from U.S. Provisional Patent Application No. 63/091,433 filed Oct. 14, 2020 and titled Pollination Predictor System and Method. The entire contents of U.S. Provisional Patent Application No. 63/091,433 are hereby incorporated by reference.

### FIELD OF THE INVENTION

This invention relates generally to technologies that allow for, and/or enable, improved crop output, such as increased harvest. In particular, this invention allows for the organization, simulation (also called modeling) and management of pollen application to maximize the biological potential of a specific seed, grain, or fruit crop. Part of any pollen application management system is identifying the most optimal stage of reproductive development for cross-pollination and selecting the best time step, such as a day, to intentionally pollinate the crop based on this optimal stage. This invention allows the user to monitor a range of measurable and/or monitorable parameters in a field, greenhouse, or controlled environment setting, including both crop parameters and other environmental parameters. By measuring and monitoring crop and environmental parameters, the user can maximize or otherwise alter crop yield, genetic purity, health, and/or composition of the seed resulting from intentional cross pollination. Accordingly, the invention provides a system that will allow users to identify the best day(s) to intentionally pollinate a particular crop in a particular location with the goal of maximizing seed yield, percent seed set, and/or influencing other crop yield characteristics. To that end, the invention increases efficiency and, in some embodiments, may provide the most efficient yield (or other maximized characteristics) in view of one or more relevant factors.

### BACKGROUND

The current invention has application to the field of pollination and crop production practices, including but not limited to seed, grain, and fruit production practices.

This invention is primarily applicable to hybrid, or varietal production, but can also be used in some non-hybrid production scenarios. While hybrid production is most often used for seed production, it may also be used for grain production. Non-hybrid production results when a plant is pollinated by pollen having the same genetic background, such as in self-pollination and sib-pollination. Hybrid plants are the result of fertilization occurring from a male pollen source of one genetic background being crossed to the female reproductive organs of a plant with a different genetic background. Hybridity among crop plants generally gives a yield advantage in commercial production and is therefore preferred, if possible, to open or self-pollinated methods of producing many commercial Poaceae crops. Crop yields began to increase markedly with the widespread introduction of hybrids in the 1940s, and crop yields have continued to increase steadily over time to the present day. In addition, large scale processes to produce higher yields of self-pollinated seeds also have significant potential value.

As will be appreciated by one of skill in the art, the practice of the invention disclosed herein will provide different benefits depending upon the nature of the crop. For crops in which hybrid production is commonplace, current methods of producing seed vary by species, but many methods typically involve the following components: (1) Planting female and male parent plants in a production block arranged in close proximity to one another; (2) locating the production block in an isolated location to reduce exposure to other unrelated or unwanted plants of the same species, and (3) imparting some form of male sterility to the female to render the female parent plants male sterile, thus avoiding the potential for self-pollination, which would ultimately contaminate the seed. Some crops have high rates of self-pollination due to pollen being released within the flower prior to the flower opening. Such crops are often bred to experience very high rates of self-pollinated seed.

Some crops do not require long isolation distances to prevent outcrossing due to the nature of the crop and its pollen-stigma interactions. In such cases, the practice of the current invention may not affect any isolation requirement but will still increase the rate of successful cross pollinations with designated male pollen and also decrease self-pollinations. This is made possible by optimizing the timing and improving the efficacy of any such pollinations. Accordingly, depending upon the crop being grown, the practice of the invention may totally or partially eliminate the need for, or reduce dependency upon any one, any two or all three of the costly and resource dependent components: planting males in proximity to females, isolation, and male sterility. Nonetheless, in some embodiments, the invention may be practiced utilizing any one, two, or all three of these components without departing from the scope of the invention. In addition, practice of the invention can assist the grower with determining the most ideal day to apply the pollen to the crop. Furthermore, the quantity of males required and the potential for lack of synchrony negatively impact total crop production outputs in conventional field planting and management scenarios. Practice of this invention overcomes both of these production limiting factors.

This invention is applicable to commodity grain production practices. For crops in which grain production is commonplace, current methods of producing grain vary slightly by species, but typically involve planting fields of the same seed variety to produce plants whose mature seeds will result in the desired grain characteristics. The plants in such fields are typically self-pollinated or sib-pollinated by neighboring plants in the same field or in nearby fields, and therefore, are not hybrids. There may be some cross pollination, however, by pollen entering from nearby fields of the same or similar species having different genetic backgrounds.

Practice of the invention can impact the production of crops in different ways, including an increase in the seed or fruit set, either by increasing of the number of seeds or fruits on the plant, or by increasing the size of the seeds, or both. In addition, the invention can impact the composition of the seed, the health of the seed, and the purity of the seed. Practice of the invention described herein will result in efficiencies, greater seed output, increased yield, and/or improvement of other desirable characteristics, including but not limited to preferred content of oil, starch, protein, and/or other nutritional components for both hybrid seed crops and self/sib-pollinated crops, whether those crops are grown for seed production, grain production, or fruit production.

### BRIEF DESCRIPTION OF THE FIGURES

FIG. 1 provides an example of an embodiment of a weather module in a computer-implemented embodiment of the present invention.

FIG. 2 provides an example of an embodiment of a plant population module in a computer-implemented embodiment of the present invention.

FIG. 3 provides an example of an embodiment of a plant population stigma exsertion module in a computer-implemented embodiment of the present invention.

FIG. 4 provides an example of an embodiment of a plant population shedding pollen module in a computer-implemented embodiment of the present invention.

FIG. 5 provides an example of an embodiment of a pollination simulation module in a computer-implemented embodiment of the present invention.

FIG. 6 provides an example of an embodiment of an intentional pollination simulation module in a computer-implemented embodiment of the present invention.

FIG. 7 provides an example of an embodiment of a logistics management module in a computer-implemented embodiment of the present invention.

FIGS. 8-12 provide examples of graphical user interfaces in a computer-implemented embodiment of the present invention.

### SUMMARY OF THE INVENTION

In a first embodiment of the method, provided is a method for pollinating a crop plant having one or more stigmas that are receptive to pollen and that produces at least one seed, grain, or fruit of interest. The method includes ingesting, as input data, material regarding reproductive maturity data for a population of the crop. The reproductive maturity data may be information that includes information sufficient to determine one or more days on which the crop plant will be receptive to pollen. Further, the method may provide a step of modeling the input data within a computing environment to identify one or more time steps during which to intentionally pollinate the population by: (1) generating the amount of receptive stigmas in the population during a plurality of time steps; (2) modeling the effect of intentionally applied pollen during each time step to transform the number of receptive stigmas during each time step into a modeled output of the seed, grain, and/or fruit of interest; and (3) generating one or more time steps during which intentional pollination is modeled to provide a greater harvest of said seed, grain, or fruit of interest than other of said time steps. The method may be a computer-implemented method. The method may be a method for pollinating the crop plant and further include intentionally pollinating the population of the crop plant during the one or more time steps during which intentional pollination is modeled to provide a greater harvest of said seed, grain, or fruit of interest than other of said time steps.

In some embodiments, the method may further model the availability of pollen for natural pollination during each time step. Such a step may include modeling the amount of available pollen during each time step and modeling the number of stigmas that are naturally pollinated during each time step. The time step may be one day. The crop may be corn. Intentionally applied pollen may be fresh pollen, persevered pollen, or both.

The reproductive maturity data maybe include one or more of (1) the amount of time needed between planting the crop and the crop beginning to exsert stigmas that are receptive to pollen; (2) the amount of heat units that are needed for the crop to exsert stigmas that are receptive to pollen; (3) the number of stigmas per plant; (4) the rate at which the crop exserts stigmas that are receptive to pollen; and (5) the number of time steps during which the crop's exserted stigmas remain receptive to pollen.

Modeling the availability of pollen for natural pollination during each time step may include ingesting data related to pollen shed including one or more of: (1) the amount of time needed between planting one or more plants that will shed pollen and those plans beginning to shed pollen; (2) the amount of heat units that are needed between planting one or more plants that will shed pollen and those plans beginning to shed pollen; (3) the amount of pollen shed from each plant; (4) the rate at which the plant sheds pollen; and (5) the number of time steps during which the crop sheds pollen.

The method may be applied to crop plants in a plurality of growing environments. Therefore, the program may generate one or more time steps for each growing environment during which intentional pollination is modeled to provide a greater harvest of said seed, grain, or fruit of interest than others of said time steps. The plurality of growing environments may be a plurality of fields in different locations. The method may generate a calendar of time steps for each growing environment during which intentional pollination is modeled to provide a greater harvest.

Pollination may be cross-pollination. In some embodiments, input data may include weather information, such as historical weather data, current day weather data, and/or forecasted weather data. Moreover, the value of the harvest may be increased by practicing the present invention.

In another embodiment of the invention, provided is another method for pollinating a crop plant having one or more stigmas that are receptive to pollen and that produces at least one seed, grain, or fruit of interest. The method includes ingesting, as input data, material regarding reproductive maturity data for a population of the crop. The reproductive maturity data may be information that includes information sufficient to determine one or more days on which the crop plant will be receptive to pollen. Further, the method may provide a step of modeling the input to identify one or more time steps during which to intentionally pollinate the population by: (1) generating the amount of receptive stigmas in the population during a plurality of time steps; (2) modeling the effect of intentionally applied pollen during each time step to transform the number of receptive stigmas during each time step into a modeled output of the seed, grain, and/or fruit of interest; and (3) generating one or more time steps during which intentional pollination is modeled to provide a greater harvest of said seed, grain, or fruit of interest than other of said time steps. The method may further include intentionally pollinating the population of the crop plant during the one or more time steps during which intentional pollination is modeled to provide a greater harvest of said seed, grain, or fruit of interest than other of said time steps.

In a third embodiment, there is provided a computer program configured to cause a processor to perform any computer-implementable method described herein, including those of the first and second embodiments above. The computer program may be a software implementation. The computer program may be provided on a computer readable medium, which may be a physical computer readable medium such as a disc or a memory device, or may be embodied as a transient signal. Such a transient signal may be a network download, including an internet download. The computer readable medium may be a computer readable storage medium or non-transitory computer readable medium.

In a fourth embodiment, there is provided a computing apparatus configured to perform any method described herein as computer-implementable, including those of the first and second embodiments above. The computing apparatus may comprise one or more processors and memory, the memory comprising the computer program of the third embodiment. The computing apparatus may be provided by a user device, such as a laptop, tablet computer or smartphone.

The computing apparatus may further comprise an input device for ingesting input data. The user input device may promise a keyboard, keypad or touchscreen. The computing apparatus may further comprising an output device for providing an indication of the selected pollination window to a user for assisting the user in performing the pollination. The output device may be display device, an audio output device, or a device for providing haptic feedback, for example.

### DETAILED DESCRIPTION

Disclosed is a unique and unprecedented system and method for simulating intentional pollen application for a particular crop in a particular location based on the complex interaction of reproductive and environmental variables. This system and method enable the user to plan for and coordinate the timing of intentional pollen application to receptive female flowers under commercial production conditions in a manner which maximizes seed or fruit yield output, genetic purity of seed or fruit produced, and/or seed quality. Seed quality may include, but is not limited to, optimizing one or more characteristics of the seed. The system and method are applicable to all crops in which intentional cross-pollination between male and female plants is a desired outcome. The system and method can also be applied to crops in which intentional pollination serves to improve overall pollination events in crops that are typically self-pollinated, which can be useful when the crop's level of pollen production is unexpectedly reduced or when other conditions threaten the success of the typical self-pollination outcome.

One of skill in the art understands that the availability of sufficient pollen, regardless of the means of delivery, is the single greatest system level factor that restricts output in agricultural crops that rely on pollination for crop outputs, such as seeds, grains, or fruits. If pollination fails, the crop fails. The availability of sufficient pollen at the correct time is critical to crop success, but can be limited by a significant number of factors in natural settings. Having the ability to introduce pollen into the system overcomes many potential shortfalls of natural pollination systems. Having the ability to introduce that pollen on the best possible day(s) (or other time step(s)) allows the grower the greatest opportunity to improve the outcome, such as harvest, of the crop. The intentionally applied pollen may be preserved or fresh, but in most embodiments it is preserved. Any preservation technique known in the art, now or in the future, may be used. Examples of preservation techniques may be found in U.S. Pat. No. 10,575,517 and United States Patent Application Publication No. 2019/0008144. The disclosures of both U.S. Pat. No. 10,575,517 and United States Patent Application Publication No. 2019/0008144 are hereby incorporated by reference in their entireties.

In one embodiment, the system and method may be carried out via an inventive software system and method. However, the invention is not limited to such an embodiment. As noted above, the system and method are applicable to all crops in which intentional cross-pollination between pollen-providing male plants and receptive stigma-bearing female plants is a desired outcome. Accordingly, the invention may be used with many plant species, whether their floral structures are designed for cross pollination, whether male sterility is imposed to ensure cross pollination, or whether their floral structures are designed for self-pollination but pollen delivery to stigmas limits seed formation. Corn, also called maize, and features of corn seed production may be discussed herein as an example only. It will be understood by one of skill in the art that other types of plants produce flowers that follow the same or a similar pattern of development to those of corn. Namely, their male and female floral components reach a functional state (referred to as anthesis) wherein application of pollen is most productive and/or efficient for seed formation as described herein below.

Growers of both hybrid and non-hybrid crops typically grow their crops in multiple fields, and often the individual fields can be quite large, including fields of 200 acres or more. Accordingly, the conditions in one field or in one portion of a large field will not be identical to the conditions in a different field, or in a different portion of a large field. The invention allows the user to determine the differences between fields or portions within fields to identify subtle differences in conditions that dictate different timing of pollen application. Accordingly, a grower can prioritize intentional pollen application among multiple fields and/or populations of plants. Furthermore, within a given population of plants, competition between plants can cause undesirable delays in a percentage of the population, resulting in their germination occurring later than other plants, or resulting in their growth being delayed, resulting in later flowering and delayed readiness for pollination. In standard field production systems, these plants become mature when pollen is no longer present and therefore remain unpollinated or poorly pollinated. By intentionally pollinating on a carefully calculated day in a specific field or part of a field with a calculated amount of pollen, the yield or composition of the target crop is significantly improved. The intentional pollination can occur on multiple occasions, providing an opportunity to better manage the variable conditions in different parts of a field or between different fields, as well as managing the variability of maturation in a given population of plants. Furthermore, growers can intentionally apply pollen using methods as described in Applicant's US patent application publication US20210059276.

In many cases, a maximum or near maximum response to intentional pollination may occur over several time steps, such as days, depending on the flowering dynamics of the species and plant population. Growers then have an opportunity to select among these days to align intentional pollinations to the day or days with weather and field conditions most favorable to the success of pollination and seed or fruit set.

These conditions include, but are not limited to:

(1) Female Plant Water Status: Conditions are most favorable when plant temperature is below air temperature indicated plants are free of water or temperature stress. Conditions are less favorable when plant temperature exceeds air temperature. Wilted plants should not be pollinated.

(2) Time of Day: There may be an optimum time of day during which pollen-stigma interactions are most favorable for pollen germination and support of pollen tube growth leading to ovary fertilization.

(3) Air Temperature: Pollen viability decreases rapidly when air temperature exceeds 32° C. (90° F.). Sensitivity to high temperature varies with species, but intentional pollination should be avoided when air temperature exceeds 35° C. Likewise, air temperature below 18° C. slow pollen germination, reducing the success of pollination.

(4) Relative Humidity: Desiccation decreases viability of recalcitrant pollen (high moisture content) species. Relative Humidity between 65% and 90% is most favorable for intentional pollination of these species. Pollination when Relative Humidity is above 95% also should be avoided due to increased potential for free water to form on the stigma surface.

(5) Vapor Pressure Deficit: Values less than 1.5 kPa are most favorable to pollination success. Values between 1.8-2.0 kPa increase risk of pollination failure. Values above 2.0 kPa should be avoided due to rapid decrease in pollen moisture in recalcitrant pollen species.

(6) Wind Speed: Mild wind movement less than 2.2 m/s (5 mph) is most favorable to intentional pollination. Intentional pollination when wind speed exceeds 5.4 m/s (12 mph) should be avoided due to increased potential for disrupting pollen-stigma contact and pollen germination.

(7) Dewfall: Free water on the surface of stigmas causes pollen to burst, preventing pollen germination. Avoid intentional pollinations when dewfall is expected after pollination or if plant tissue are wet from dew.

(8) Rain in the Forecast: The favorable time interval between intentional pollination and a rainfall event depends on the species and time required for pollen rehydration and pollen tube entry into the stigma body. Rainfall sufficient to moisten reproductive structures within 60 min of pollination increases risk of pollination failure. Avoid intentional pollination if rain is expected within 15 minutes or if plants have not dried from a previous rainfall.

Intentional pollination also provides an alternative to conventional insect-dependent production systems. Many insect-based production systems are experiencing significant challenges due to and other pressures, including colony collapse disorder in bees. These problems have caused significant declines in insect populations. The opportunity to intentionally apply pollen rather than rely upon currently challenged insect-based systems allows growers an opportunity to improve crop production. Moreover, the simulation of the present method is applicable to such crops.

Furthermore, climate change is increasingly causing unusual weather conditions or extreme weather events that significantly impact agricultural practices. Unusual temperatures can affect when plants germinate, their growth rates, and when they produce pollen or flowers. Unusual storms and severe weather events can impede plant growth or cause crop damage that affects production. As such events and weather fluctuations become more commonplace, there is a greater potential for crop failure or severely impacted crop output. The present invention allows growers to conduct simulations following unusual and unexpected weather events to determine how supplemental intentional pollinations may help with crop recovery or “rescue” a field that would otherwise be non-productive. By simulation of preserved or freshly collected pollen application, the grower can determine the best day(s) for such an application to maximize the potential crop output in spite of the challenges posed by weather extremes.

Plant reproductive systems are complex, and many variables influence the timing of maturity for both the male plants or male plant components and the female plants or female plant components. Because many variables impacting reproductive maturity are not controlled, growers rarely achieve optimal crop output. This situation is complicated by growers having to account for variables across fields in different locations, each with separate microclimates and both biotic and abiotic factors that render each field different and sections within fields different. In addition, modern agriculture has introduced many adaptations in crop plants that have resulted in those plants struggling to support the demands placed upon them in a commercial system. The stresses placed upon plants in commercial agricultural systems are significant, including those posed by weather, pests, diseases, population density, inadequate soil, and other factors, cause the plants to divert energy from pollen production and/or development of reproductive structures, and may influence overall reproductive health. See, e.g. Duvick, D. N. 1997. What is yield? p. 332-335 In G. O. Edmeades, B. Banziger, H. R. Mickelson and C. B. Pena-Valdivia (ed.) Developing Drought and Low N-Tolerant Maize. Proceedings of a Symposium, Mar. 25-29, 1996, CIMMYT, El Batan, Mexico. CIMMYT, Mexico, D. F.; Bastos, L. M., W. Carciochi, R. P. Lollato, B. R. Jaenisch, C. R. Rezende, R. Schwalbert, P. V. V. Prasad, G. Zhang, A. K. Fritz, C. Foster, Y. Wright, S. Young, P. Bradley, and I. A. Ciampitti. 2020. Winter Wheat Yield Response to Plant Density as a Function of Yield Environment and Tillering Potential: A Review and Field Studies. Front. Plant Sci., 5 Mar. 2020. https://doi.org/10.3389/fpls.2020.00054; Gonzalez, V. H., E. A. Lee, L. N. Lukens, and C. J. Swanton. 2019. The relationship between floret number and plant dry matter accumulation varies with early season stress in maize (Zea mays L.). Field Crops Res. 238: 129-138. https://doi.org/10.1016/j.fcr.2019.05.003; Saini, H. S., and M. E. Westgate. 1999. Reproductive development in grain crops during drought. Adv. Agron. 68: 59-96. https://doi.org/10.1016/S0065-2113(08)60843-3; Manju, L. G., T. Mohapatra, A. S. Geetanjali, K. R. S. S. Rao. 2017. Engineering Rice for Abiotic Stress Tolerance: A Review. Current Trends Biotech. Pharm. 11: 396-413; Irenaeus, K. S. T., and S. K. Mitra. 2014. Understanding the pollen and ovule characters and fruit set of fruit crops in relation to temperature and genotype—a review. J. Appl. Bot. Food Qual. 87: 157-167. https://DOI:10.5073/JABFQ.2014.087.023; Fischer, G., F. Ramirez, and F. Casierra-Posada. 2016. Ecophysiological aspects of fruit crops in the era of climate change. A review. Agronomia Colombiana 34: 190-199. http://dx.doi.org/10.15446/agron.colomb.v34n2.56799.

Managing the timing and priority for intentional pollination across a total number of fields, each with slightly different conditions and different requirements, can be extremely difficult for the grower given all the different inputs and variables. Before the advent of this invention, growers relied upon management best guesses and natural pollination mechanisms. The present invention provides a system that gives a clear prioritization scheme to the grower and allows them to manage the timing of intentional pollinations in an organized manner. Such a system has never before been available to growers.

One method currently used by growers to try to react to the unpredictability of plant reproductive outcomes is to overproduce seed supplies. This is intentionally done in order to offset product losses due to pollination failures, which invariably occur each season. This is symptomatic of a system that is far from optimal. The present invention allows growers to avoid the need for overproduction by overcoming the many variables impacting pollination and allowing the grower to know the right day(s) to intentionally pollinate the right crop in the right location. There is no longer a need for the grower to depend on achieving perfect natural timing between natural pollen availability and optimal female receptivity (anthesis synchrony). Stored, preserved pollen or the ability to collect fresh pollen from a different location immediately prior to application at another location allows the grower to always pollinate to maximize desired output.

In some embodiments, methods of the present invention are applicable to a crop which includes a population of plants, which is defined as 50 or more plants, such as a field of plants, or a population of plants growing in a hydroponic facility, vertical farming facility, or other growing environment. A population of plants may include plants having one, two, three, or more genetically distinct backgrounds. In some embodiments, the methods are applicable to a field of plants. A field may be any size but is typically at least 1/10 of an acre and may be any size above 1/10 of an acre. Common field sizes in the United States are between 40 acres and 200 acres. Fields in other areas of the world may be smaller or larger. Accordingly, a field may be, but is not limited to, 1/10 acre, ⅕ acre, 3/10 acre, ⅖ acre, ½ acre, ⅗ acre, 7/10 acre, ⅘ acre, 9/10 acre, 1 acre, 2 acres, 3 acres, 4 acres, 5 acres, 6 acres, 7 acres, 8 acres, 9 acres, 10 acres, 11 acres, 12 acres, 13 acres, 14 acres, 15 acres, 16 acres, 17 acres, 18 acres, 19 acres, 20 acres, 25 acres, 30 acres, 35 acres, 40 acres, 45 acres, 50 acres, 55 acres, 60 acres, 65 acres, 70 acres, 75 acres, 80 acres, 85 acres, 90 acres, 95 acres, 100 acres, 105 acres, 110 acres, 115 acres, 120 acres, 125 acres, 130 acres, 135 acres, 140 acres, 145 acres, 150 acres, 155 acres, 160 acres, 165 acres, 170 acres, 175 acres, 180 acres, 185 acres, 190 acres, 195 acres, 200 acres, 205 acres, or 210 acres. An acre may be defined as approximately 4047 square meters. It will be understood by one in the art that the amount of time necessary to intentionally pollinate a field will depend on many factors including, but not limited to, field size, the speed at which pollination may occur, and the number of people and/or devices available to pollinate. For example, a field of 40 acres may take approximately 2.5 hours to intentionally pollinate.

This invention provides an improved method of identifying the best day(s) to intentionally pollinate a field, a portion of a field, or multiple fields, by intentionally applying male pollen to flowers on female plants. The conventional layout of any given field which relies on cross-pollination must reflect its dependency on natural pollination. The presence of any plant in such a field which is solely present as a pollen donor is a direct reduction to female yield in the natural system. Practice of the present invention introduces the never-before available opportunity to plan field layouts in a way which focuses on yield with the knowledge that a prescriptive, intentional pollination will occur at one or more timepoints that provide the most value. In some cases, no male plants are required within the layout, as pollination with preserved pollen reduces or eliminates the need for males to be actively shedding pollen within the field. Practice of the present invention also has the potential to significantly add value to the crop output in a number of ways, including, but not limited to, the potential for higher yields which result in higher value sales; the potential for improved crop characteristics, which enable the crop output to be sold for a higher value; and the increased efficiency per unit of land, which provides a cost savings and thereby adds value to the crop.

Use of the term “intentional” with regard to pollen application means the specific application of pollen in a way that does not include, or exclusively involve, natural pollination by wind, insect activity, or other naturally-occurring conditions. Intentionally applied pollen is pollen that has been applied to a plant as a result of a deliberate human activity, decision, or intervention, and may be applied by hand or by other means. In some embodiments, the intentional release of pollen may include releasing pollen in proximity to said crop to be pollinated such that said pollen is capable of pollinating said crop. For the purposes of this invention, the term “preservation” or “preserved pollen” means any storage of collected pollen that results in a level of viability, fertility, or both, which is different than the level of viability, fertility or both, which would occur if the pollen were held in uncontrolled conditions. This invention may use preserved pollen at any time, including but not limited to when the selected pollination day is outside the period of time during which pollen is normally shedding. The preserved pollen may be pollen that has been frozen, chilled, mixed with other particles or liquids, or otherwise treated to preserve its longevity and viability. Preservation may include conditioning steps immediately upon harvesting the pollen to retain or improve its longevity or viability. Methods used may include, for example, those described in U.S. Pat. No. 10,575,517 or US patent application US20190008144, the entire disclosures of which are hereby incorporated by reference. Preserved pollen may have been preserved by any means that permits the pollen to have the necessary level of viability for the application, including but not limited to various forms of cooling or freezing including, but not limited to, chilling, cryopreservation, freeze drying, storage with selected additives to prolong viability, or storage in liquid nitrogen.

By intentionally delivering, releasing, and/or applying pollen on the day(s) determined by practicing the invention, and for at least a portion of the duration of a plant's fertility period when the plant is receptive to pollen or when the environmental conditions are favorable to the success of pollination, the seed set, fruit set, yield, and/or other desirable characteristics including but not limited to preferred content of oil, starch, protein, and/or other nutritional components can be enhanced, improved, changed, minimized, and/or maximized over that which would have been obtained by relying on natural pollination. However, one of skill in the art will also recognize that the duration of pollen delivery, release, and/or application may also operate on a continuum to achieve varying levels of seed and fruit set. Pollen delivery may be for the entire duration of a plant's fertility period or a portion of the duration of a plant's fertility period. Pollen delivery may occur one or more times per day and/or one or more times per fertility period. Pollen can be delivered, released, and/or applied in any number of ways, including, but not limited to manually, manually with a small hand mechanical device for semi-automated dispersal, by field driven machinery containing pollen dispersal machinery, or via fully automated dispersal by a self-propelled and/or human guided apparatus such as a drone that has a pollen dispersal device mounted to it, wherein the pollen dispersal is by automatic means, including, but not limited to, mechanical or pneumatic means.

Use of a drone would be especially novel and practical in this method. In one estimation, 450 grams (approximately 1 lb) of pollen is more than sufficient, when directed to exposed receptive silks, to pollinate 8 hectares (approximately 20 acres) of female corn plants. This is calculated as follows: 4 pollen grains/receptive floret×500 florets/rachis×1 rachis/plant×26,000 plants/ac×20 acres×275 ng/pollen=286 grams of pollen. This would allow small drones, which need not be regulated, to be used in the method and which can be guided using GPS coordinates to focus the pollen dispersal directly over the female plants. When the ideal pollination day has been identified, the drones are released to conduct the pollinations in the target crop population. The drones can be activated manually, or in other embodiments, the drones can be activated by signals received from a weather station or other device at the time that the ideal pollination day has been identified and correlated with the time it will take the drones to pollinate the size of the field and the number of plants in the plant population. The drones may need to be refilled with pollen when the field is of a sufficient size.

This invention can operate in any crop plant (as noted above) to improve output. It can operate in any environment including, but not limited to, ideal or target growing environments, off-season environments, or controlled environments (e.g., shade house, glass house, greenhouse, hoop house, growth chambers, vertical farming facilities, hydroponic facilities, aeroponic facilities, etc.).

The system and method of the invention may use one or more factors to help the user determine the best pollen application time step(s), such as day(s). It is understood that the system can be used by doing manual calculations to determine the optimal pollen application criteria, or the system can be automated by software or other means, wherein the calculations are completed for the user when parameters are used as inputs. Alternatively, a combination of manual and computer-implemented methods may be used. The parameters that can be used in the calculations, whether manual or not, include but are not limited to one or more of the following: (1) Reproductive plant maturity data based on plant developmental characteristics may be recorded and/or may be input into the system over one or more occasions. Data may include, but are not limited to, one or more of: Percentage of plants in a population exserting receptive stigmas from female reproductive structures; (2) Percentage of plants in a population releasing pollen from male reproductive structures; (3) Density of pollen production; (4) Pollen viability; (5) Duration of time unpollinated flowers remain receptive to pollen; (6) Duration of the time during which pollen is released from male plants; and/or (7) Duration of stigma exsertion on female plants

Daily weather data may be tracked and/or input into the software/system to provide additional information to predict progress of plant development or condition of the male and female components to affect fertilization. Data may include, but are not limited to, one or more of: (1) Heat unit accumulation. This is typically measured in growing degree day units which impact the rate of development and plant biomass accumulation; (2) Precipitation; (3) Air temperature; (4) Total sunlight; (5) Relative humidity and/or vapor pressure deficit; and/or (6) Wind speed

Soil metrics may be input into the software/system and utilized to provide additional information to predict progress of plant development based on the soil's physical and/or chemical characteristics. This may include, but is not limited to, one or more of: (1) NRCS soil classification; (2) Nutrient composition; and/or (3) Water holding capacity

Agronomic data regarding plant morphological characteristics and development rates for specific plant genotypes available in public and/or private sector databases may be input into the software/system to assist in predicting the plant growth rates, pollen shed density, pollen shed duration, stigma exsertion rates, and/or average days to reproductive maturity in a given geographical region during specific dates of the year. Such a database may be part of a system and/or method of the present invention. Furthermore, other databases exist which can be used by the system, such as databases owned by seed or fruit production companies and/or other parties. A system and/or method of the present invention may use data from any database, including data gathered from field notes, yield trials, visual observations, RGB images, LIDAR, satellite imagery, radar, and sonic sensing. In some embodiments, the software/system may accumulate this information to create and/or add to its own database to use in the future.

The system may consider data related to sterility, including but not limited to male sterility and/or chemical sterility.

The inventive method and system may include one or more of the following tasks utilizing the data listed above: (1) Calculate the percentage of receptive female flowers that will be pollinated each day under natural pollination conditions. Natural pollination conditions exclude the application of pollen, including but not limited to preserved pollen; (2) Calculate when the timing and intensity of naturally shedding pollen will be a limiting factor for pollination in comparison with population of receptive female flowers; (3) Calculate when the population of unpollinated female flowers will peak in number and receptivity to maximize the output of seed or fruit and/or other desirable seed characteristics resulting from the application of an external source of pollen; and/or (4) Combine with weather forecast data to provide information on the day on which intentionally applied pollen will have the greatest impact for seed, grain, or fruit yield, genetic purity, and/or seed quality.

In one or more embodiments of the invention utilizing a software program, the program may be designed to include one or more of the features described below. In those features, one or more variables/inputs may be provided as an internal constant, input by a user, or calculated by the software.

As discussed above, in one or more preferred embodiments of the invention, the method may be computer-implemented. Such a method may include one or more types of input data coupled with modeling, also called simulation, output. The simulation output is the result of one or more calculations. At its highest level, a computer-implemented method of the present invention identifies the amount of the crop that is receptive to pollen over time to determine the time step(s), such as a day or days on which intentional pollination will result in a greater harvest compared to other time steps, such as other day(s) The receptivity to pollen is transformed via the method to a simulated harvest. Increased harvest is defined by the user for a given situation and may include, but is not limited to, increased yield, increased purity, increased desirable characteristics, and/or decreased undesirable characteristics. In one or more embodiments, greater harvest may be a simulated seed set.

A preferred embodiment of a computer-implemented method of the present invention is described in detail below. The description identifies a plurality of modules and user interface screens. However, one of skill in the art will understand that any number of modules and user interfaces may be used without departing from the scope of the invention.

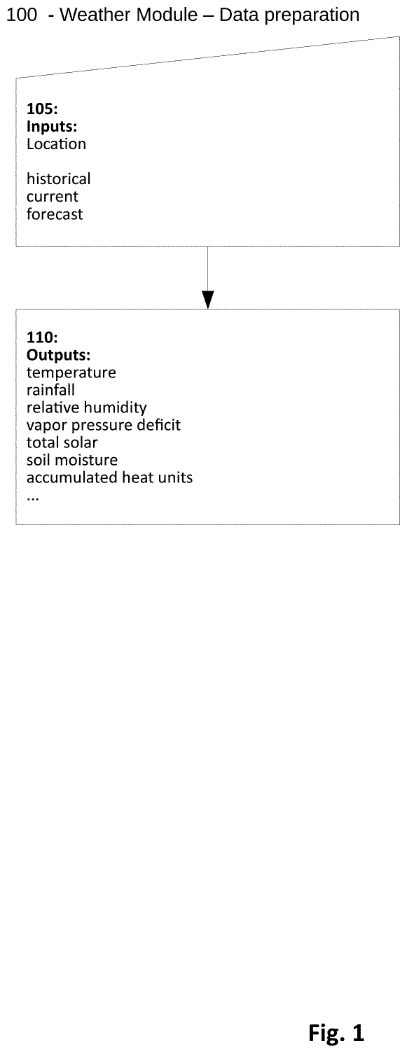

Referring to FIGS. 1-7, a plurality of modules of the present invention are described. Referring first to FIG. 1, a Weather Module is shown. The weather module may include one or more inputs and calculations, also called outputs. Input related to the weather module may be from any source, such as from a user or from a third-party source. For example, weather input may be from an online source, a governmental database, or from weather station hardware placed in or near the location. In preferred embodiments, the weather module pulls this information, from any source, to create outputs that serve as inputs in other modules. Furthermore, definitions of the inputs and outputs of an Exemplary Weather Module are in Tables 1 and 2.

TABLE 1
Definitions of the Inputs of an exemplary Weather Module.

| | |
|---|---|
| Location | The location of the population of plants. In preferred |
| | embodiments, location may be inputted in latitude/ |
| | longitude inputs or GPS coordinate input. |
| Historical | Data related to historical weather conditions for the |
| Weather | location. |
| Information | |
| Current Day | Data related to the current weather conditions on a day |
| Information | when the method is being used. |
| Weather | Data related to the weather forecast on a day when the |
| Forecast | method is being used. |

TABLE 2
Definitions of the out puts from an exemplary weather module
that may serve as inputs in other areas of the method.

| | |
|---|---|
| Temperature | A measure of the warmth or coldness of the air |
| | temperature at the location. |
| Rainfall | A measure of the amount of rainfall, if any, at the |
| | location. |
| Relative | A measure of the amount of water vapor in the air, |
| Humidity | expressed as a percentage of the maximum amount that |
| | the air could hold at the given temperature. |
| Vapor | A measure of the difference between the amount of |
| Pressure | moisture in the air and how much moisture the air |
| Deficit | can hold when it is saturated. |
| Total | Total Incident Solar Radiation is the amount of solar |
| Solar | radiation that hits the earth's surface per unit |
| | time and area. Typical units are watts/m\hat{ }2 *sec). Total |
| | Incident Photosynthetically Active Radiation (IPAR) |
| | is the component of Total Solar from 400 to 700 nm |
| | wavelengths that is active in promoting photosynthesis. |
| | Typical units are μmoles/m\hat{ }2*sec. In both cases, |
| | values relevant to plant development typically are |
| | summed per day, plant growth stage, crop growth |
| | interval, or growing season. |
| Soil | The amount of water contained within a specific mass |
| Moisture | or volume of soil. Typical units are grams/kg of soil, |
| | liters/kg soil, mm/meter soil, inches/foot soil, and |
| | percent moisture (g/g*100). The amount of soil moisture |
| | available to plants is determined by the degree of |
| | saturation and inherent water holding capacity of the |
| | soil matrix. |
| Accumulated | A measure of the cumulative effect of temperature |
| Heat Units | over time. |

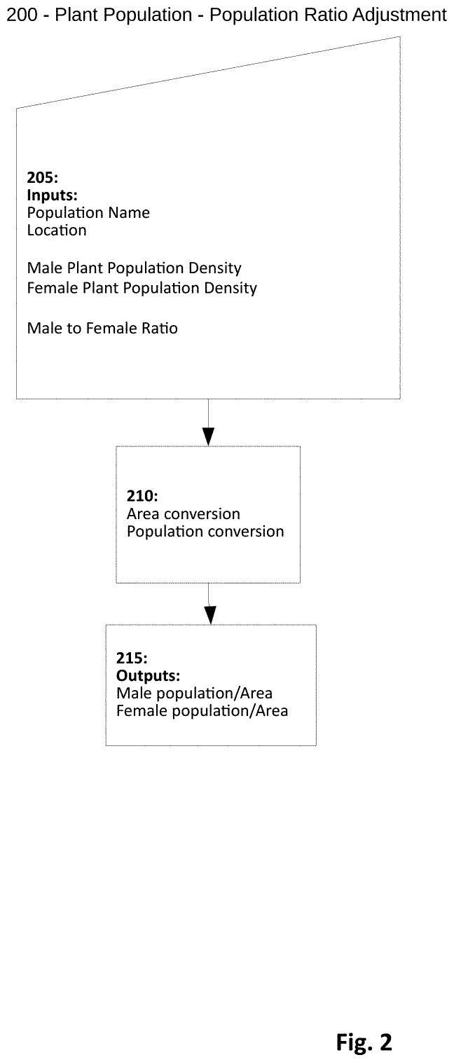

Referring to FIG. 2, a Plant Population Module provides a module with inputs and outputs/calculations related to the population of plants. Specifically, this is a place where a user can input information related to the growing environment and the plants therein for use in other modules of the method. This module then performs calculations to create output that becomes input in downstream modules. Definitions of the Inputs, Calculations, and Outputs of the Plant Population Module are found in Tables 3, 4, and 5, respectively.

TABLE 3
Definition of Inputs in the Plant Population Module.

| | |
|---|---|
| Population | The name given to a particular population of plants by |
| Name | a user. |
| Location | The location of the population of plants. In preferred |
| | embodiments, location may be inputted in latitude/ |
| | longitude inputs or GPS coordinate input. |
| Plant | The number of female plants per unit area intended for |
| Population | seed, grain, or fruit production. This input assumes |
| Density | that each plant can produce one or more seeds, grains, |
| | and/or fruit, as applicable. This input is variable and |
| | typically inputted by a user at the beginning of a |
| | season. This input may be adjusted in-season to correct |
| | for low germination or stand loss. Separate plant |
| | population density inputs are used for each population |
| | of female plants. |
| Male | Number of plants/area shedding pollen. |
| Plant | |
| Population | |
| Density | |
| Female | Number of plants/area exserting silks |
| Plant | |
| Population | |
| Density | |
| Male to | A ratio of female plant to male plants used to adjust |
| Female | the planted population density of female and male |
| Ratio | plants to an effective plant population density for |
| | calculating the actual pollen shed density and number |
| | of receptive stigmas per area. This ratio can be |
| | determined by, but is not limited to, the following |
| | user inputs: male and female row numbers, relative |
| | planting densities, distance between male and female |
| | plants, and prolificacy of male and female flowers on |
| | individual or neighboring plants. |

TABLE 4
Definitions of the Calculations of the Plant Population Module.

| | |
|---|---|
| Area | Adjust the input of area to Metric units (Hectare) for |
| Conversion | further calculation consistency. |
| Population | Adjust the input of area to Metric units (Hectare) for |
| Conversion | further calculation consistency. |

TABLE 5
Definition of Outputs of the Plant Population Module.

| | |
|---|---|
| Male Population/Area | The number of male plants/area |
| Female Population/Area | The number of male plants/area |

The method may generate the best day or days for pollination resulting in a greater harvest by analyzing input related to the crop, including but not limited to, the type of crop, the location of the crop, and information regarding the reproductive maturity of the crop. In some embodiments, the method will simulate the number of receptive stigmas each day; the available natural pollen, if any, on a given day; the number of receptive stigmas pollinated naturally, if any; the number of receptive stigmas that would be pollinated with intentional pollination; and the harvest resulting from intentional pollination on a particular day.

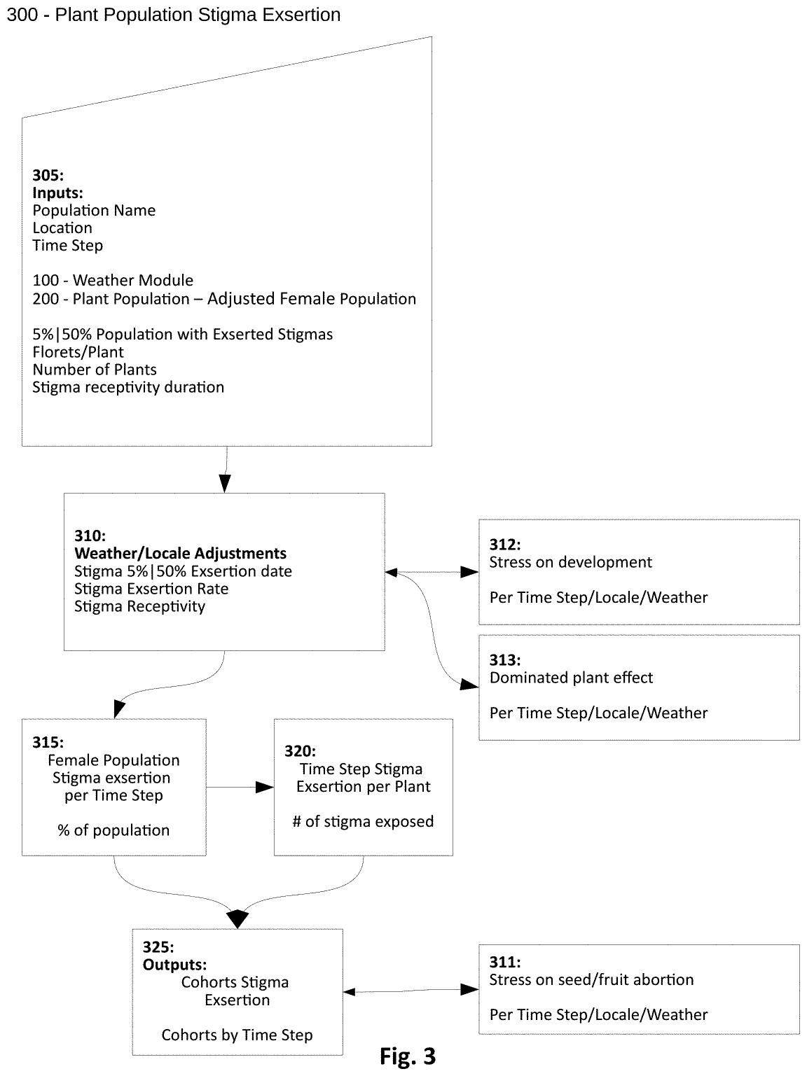

Accordingly, preferred embodiments of the invention include one or more plant stigma modules to receive input and perform calculations related to the stigmas of a crop that are receptive to pollen. Those stigmas are pollinated to produce the seed, grain, or fruit of interest. Referring to FIG. 3, an exemplary embodiment of a plant population stigma exsertion module is shown. The module may include several inputs in a first step 305 that may then be adjusted based on factors relevant to stigma exsertion in a second step 310. The adjustments of block 310 may further be adjusted as shown in blocks 311, 312, and 313 in association with stress on seed/fruit abortion, stress on development, and dominated plant effect, respectively. Further material related to these adjustments is found in Table 15. The output of blocks 310, 311, 312, and 313 is female population stigma exsertion per time step 315 and time step stigma exsertion per plant. 320 Outputs 325 of the plant population stigma exsertion module include cohort stigma exsertion and cohorts by time step, which may be used as input in other modules, as described herein below. In some software embodiments of the invention, the software will be designed to cover one crop only, such as the exemplary embodiment shown in FIGS. 1-7. In other embodiments, the software may be designed to cover multiple crops, and the user will input or select the desired crop or plant type.

As noted above, the plant population stigma exsertion module receives input related to the population of plants, including, but not limited to: the chosen time step, which often is a day, the population name (example field 1, field 2, south field, greenhouse A, etc.), and the location of the growing environment, as discussed above. Typically, this information is inputted for a plant by the user, such as during the planting process. The user may also input the expected time when 5% of the population will have exserted stigmas, along with a stigma exsertion rate. This material is typically provided to growers by the seed company from which the seed for the crops is obtained. Moreover, inputs into this module are derived from previous output from the Weather Module 100 and the Plant Population—Population Ratio Adjustment module 200 (also called the Plant Population Module). The Plant Population Module provides the Plant Population Stigma Exsertion Module with an adjusted female population and the expected dates on which 5% and 50% of the plant population will have exserted stigmas. These inputs are used to calculate one or more of include cohort stigma exsertion and cohorts by time step. Accordingly, this module is directed to a calculation that, based on the described inputs, generates the total number and percentage of plants in a population that start exserting receptive stigmas from female reproductive structures for a given time step. The time step length is typically one day but may be shorter or longer.

Definitions of the Plant Population Stigma Exsertion Inputs, Calculations, and Outputs are found in tables 6, 7, and 8 below.

TABLE 6
Definitions of Inputs in the Plant Population Stigma Exsertion Module.

| | |
|---|---|
| Time Step | The incremental change in time for which the |
| | method simulates pollination activities. |
| | Example: one day. |
| Population Name | The name given to a particular population of |
| | plants by a user. |
| Location | The location of the population of plants. In |
| | preferred embodiments, location may be inputted |
| | in latitude/longitude inputs or GPS coordinate |
| | input. |
| 100 - Weather Module | One or more of the Weather Module outputs |
| | described herein. |
| 200 - Plant Population - | One or more of the Plant Population outputs |
| Adjusted Female | described herein, particularly as it relates to |
| Population | female plants. |
| 300 - Plant Population 5% | The day when 5% of the population of female plants |
| of Stigma Exsertion | is expected to have started exserting receptive |
| Expected | stigmas (anthesis). This input is typically, but |
| | not limited to, accumulated heat units from planting |
| | to flowering based on user experience. Values are |
| | unique to species, genotype, geographical location, |
| | and environmental conditions. |
| 300 - Plant Population | The day when 50% of the population of female plants |
| 50% of Stigma Exsertion | is expected to have started exserting receptive stigmas |
| Expected | (anthesis). This input is typically, but not limited to, |
| | accumulated heat units from planting to flowering based |
| | on user experience. Values are unique to species, |
| | genotype, geographical location, and environmental |
| | conditions. |
| Florets/Plant | Average number of florets per plant that produce |
| | receptive stigmas. In some embodiments, the maximum |
| | number of florets per plant may be used. |
| Stigma Exsertion Duration | The number of days required for a typical female plant |
| | to exsert 95% of its receptive stigmas |
| Number of Plants | The number of plants included in each time step cohort |
| | based on Plant population density and Daily Percentage |
| | of Plants Exserting Stigmas. |
| Stigma Receptivity | The number of days after stigmas are first exserted for |
| Duration | pollination that they remain receptive to pollen if |
| | not pollinated. |
| Population Stigma | Slope of the equation governing the rate at which new |
| Exsertion Rate | plants are added to the female plant population to start |
| | exserting receptive stigmas. |

TABLE 7
Definitions of Calculations in the Plant Population Stigma Exsertion Module.

| | |
|---|---|
| Weather/locale adjusted | Adjustment of expected 5% exsertion date for a population due |
| Stigma 5% exsertion date | to weather stressors as can be modeled using, but not limited to, |
| | ‘Stress on development’ and ‘Dominated plant effect’ to predict |
| | the adjusted 5% exsertion date from weather impact. |
| Weather/locale adjusted | Adjustment of expected 50% exsertion date for a population due |
| 50% exsertion date | to weather stressors as can be modeled using, but not limited to, |
| | ‘Stress on development’ and ‘Dominated plant effect’ to predict |
| | the adjusted 50% exsertion date from weather impact. |
| Weather/locale adjusted | Adjustment of population standard exsertion rate, altered by time |
| stigma exsertion rate | step environmental stressors on a given population cohort. |
| | Stressors include but may not be limited to those modeled by |
| | ‘Stress on seed fruit Abortion’, ‘Stress on development’ and |
| | ‘Dominated plant effect’. |
| Weather/locale adjusted | Adjustment of standard exsertion duration altered by |
| stigma exsertion duration | environmental stressors on a given plant or timestep cohort. |
| | Stressors include, but not limited to, those modeled by ‘Stress on |
| | development’ and ‘Dominated plant effect’. |
| Weather/locale adjusted | Adjustment of standard exserted stigma receptivity altered by |
| stigma exsertion | environmental stressors on a given plant or timestep cohort. |
| receptivity | Stressors include, but not limited to, those modeled by ‘Stress on |
| | development’ and ‘Dominated plant effect’. |
| Female population stigma | Measured in percent of the population. |
| exsertion per time step | |
| Time Step Stigma | Measured in number of stigmas exposed. |
| Exsertion per Plant | |
| Number of Plants | The number of plants beginning to exsert stigmas in the female |
| Exserting Stigmas | plant population. This number is calculated for each time step, |
| | which in the preferred embodiment is each day. |
| Percentage of Plants | The ratio of plants beginning to exsert receptive stigmas to the |
| Exserting Stigmas | total number of plants in the female plant population. This value |
| | is calculated for each time step, which in the preferred |
| | embodiment is each day. |
| Temporal Dynamic of | Temporal profile accumulating from 0% to 100% of the female |
| Female Plant Population | plant population expected to begin exserting receptive stigmas. |
| Exserting Stigmas | This relates to the development of a crop based on the calendar |
| | date in which it is planted. For example, a crop planted in May |
| | might develop exserted stigmas at a different rate the same |
| | thancrop planted in June. Moreover, temporally-temporalbased |
| | plantings may serve to isolate fields or groups of plantsisolations |
| | as it relates to pollen shed. |
| Stigma Exsertion Rate | The slope in the equation determining initial rate at which |
| | stigmas are exserted by an average female plant. |
| Exsertion Intercept | The time interval at which stigma exsertion per plant starts |
| | within the calculation. |
| Daily Stigma Exsertion per | The number of newly exserted stigmas per plant each day. |
| Plant | Variation in daily intensity is determined by total florets/plant, |
| | stigma exsertion rate, and stigma exsertion duration. |
| Temporal Dynamic of | The temporal profile of stigmas exserted per plant. This profile |
| Stigma Exsertion per Plant | of daily values is calculated from the first day stigmas are |
| | exserted for pollination until all viable florets exsert stigmas. |
| Number of stigmas | The cumulative number of stigmas exserted for all plants within |
| exserted per cohort | a plant or timestep cohort. Cohorts are added at all time |
| | intervals until stigma exsertion duration is reached. Cohort |
| | values for stigmas available for pollination decreases to 0.0 |
| | when unpollinated stigmas reach the limit of stigma receptivity |
| | duration. |
| Number of stigmas | The daily summation of receptive stigmas exserted for |
| exserted for all cohorts per | pollination for all cohorts with plants actively exserting stigmas. |
| time step | This value is used in the calculation of Percent of Stigma |
| | Pollinated for each time step, which converts the total of |
| | receptive stigmas/area to seeds or fruits as described herein |
| | below. |
| Temporal population | The cumulative profile of exserted stigmas per area for the |
| dynamic of exserted | female plant population. |
| stigmas | |

TABLE 8
Definitions of Output of the Plant
Population Stigma Exsertion Module

| | |
|---|---|
| Female Population Stigma | The percent of the female population |
| Exsertion per Time Step | initiating stigma exsertion per timestep. |
| Cohorts by Time Step | LevelsLevels of cohorts representing |
| | stigma exsertion over time. |

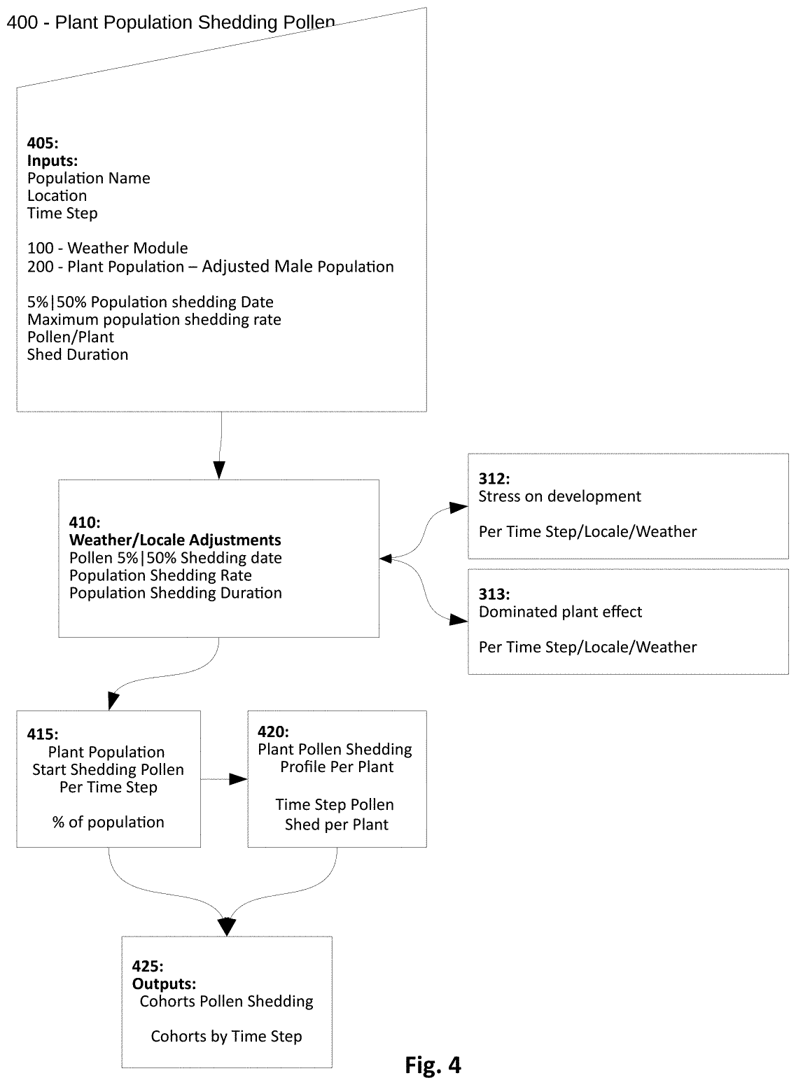

Referring to FIG. 4, the method may also include a Plant Population Shedding Population Shedding Pollen Module. As will be understood by one of skill in the art, the method of the present invention may be used with populations that include female plants or female components of plants only. Alternatively, the method of the present invention may be used with populations that include male plants or male components of plants that will shed pollen. FIG. 4 describes an example of a Plant Population Shedding Pollen Modules. In some cases, there may be no pollen in certain locations, so the outputs (which will be discussed herein below) may be zero.

As shown in FIG. 4, the exemplary embodiment of this module includes the following inputs that are typically inputted by a user: the time step, population name, and location. The user may also input the expected time when 5% or 50% of the population are expected to begin shedding pollen. This information is typically provided to growers by the seed company from which the seed for the crops is obtained. Also includes as input are outputs from the Weather Module 100 and Plant Population Module 200. Further inputs, which may come from the user, are the amount of pollen shed per plant, pollen shed duration, the cohort name, the number of plants shedding. With respect to the cohort name, methods of the preferred embodiment categorize the total number of plants capable of shedding pollen into subgroup, called cohorts. Plants that begin to shed pollen during the same time step are a cohort. Furthermore, the plants that are shedding pollen during a particular time step (regardless of when the plants begin shedding pollen) are also a cohort, and more specifically, the cohort of plants that are shedding pollen during a particular time step.

In block 410 of FIG. 4, several inputs are adjusted based on the Weather Module 100 and/or based on the location. Moreover, the inputs may be adjusted based on stress on development, dominated plant effect, or pollen viability as shown in blocks 312 and 313, respectively. Further information regarding these blocks are found in Table 15. Blocks 410, 312, and 313 flow to provide in blocks 415 and 420, plant population level calculations. Ultimately, the resulting output 425 is the Cohorts Pollen Shedding and the Cohorts by Time Step.

The definitions of the inputs, calculations, and outputs of the Plant Population Shedding Pollen Module are in tables 9, 10, and 11 below.

TABLE 9
The definitions of the Plant Population Shedding Pollen Module inputs.

| | |
|---|---|
| Time Step | The incremental change in time for which the method simulates |
| | pollination activities. Example: one day. |
| Population Name | The name given to a particular population of plants by a user. |
| Location | The location of the population of plants. In preferred embodiments, |
| | location may be inputted in latitude/longitude inputs or GPS |
| | coordinate input. |
| 100 - Weather Module | One or more of the Weather Module outputs described herein. |
| Output | |
| 200 - Plant Population | One or more of the Plant Population outputs described herein, |
| Module Output | particularly as it relates to male plants. |
| 400 - Plant Population | The date/time when 5% of the plants in this population are expected |
| 5% of Plant Population | to start shedding pollen (anthesis). This input is typically, but not |
| Shedding Pollen | limited to, accumulated heat units from planting to flowering based |
| Expected | on user experience. Values are unique to species, genotype, |
| | geographical location, and environmental conditions. |
| 400 - Plant Population | The date/time when 50% of the plants in this population are |
| 50% of Plant | expected to start shedding pollen (anthesis). This input is typically, |
| Population Shedding | but not limited to, accumulated heat units from planting to flowering |
| Pollen Expected | based on user experience. Values are unique to species, genotype, |
| | geographical location, and environmental conditions. |
| Maximum Population | The slope of the equation governing the rate at which new plants are |
| Shedding Rate | added to the male plant population to start shedding pollen. |
| The amount of | Average number of pollen grains produced per plant. Separate |
| Pollen/Plant | inputs each male population. Values are unique to species, |
| | genotype, geographical location, and environmental conditions. |
| Pollen Shed Duration | Expected period of time that a given plant is expected to shed |
| | pollen to a maximum percentage of all pollen produced by the plant. |
| Number of Plants | The number of plants per area planted as a source of pollen. This |
| | input also can be referred to as planting density, plant population, |
| | plant density, seeding density, plants per acre, or plants per unit |
| | area. |
| Pollen Viability | Maximum percentage of pollen shed that is capable of germinating |
| | on a receptive stigma and fertilizing a female flower. |

TABLE 10
The calculations of the Plant Population Shedding Pollen Module.

| | |
|---|---|
| Weather/Locale | Adjustment of expected 5% population shedding start date due to |
| Adjusted 5% of Plant | environmental stressors as modeled using, but not limited to, ‘Stress |
| Population Shedding | on development’ and ‘Dominated plant effect.’ effect’ |
| Pollen Expected | |
| Weather/Locale | Adjustment of expected 50% population shedding start date due to |
| Adjusted 50% of Plant | environmental stressors as modeled using, but not limited to, ‘Stress |
| Population Shedding | on development’ and ‘Dominated plant effect.’ effect’ |
| Pollen Expected | |
| Weather/Locale | Adjustment of standard shedding rate per plant or timestep cohort. |
| Adjusted Population | Environment stressors include, but not limited to, those modeled by |
| Shedding Rate | ‘Stress on development’ and ‘Dominated plant effect’. |
| Weather/Locale | Adjustment of standard shedding duration per plant or timestep |
| Adjusted Population | cohort. Environment stressors include, but not limited to, those |
| Shedding Duration | modeled by ‘Stress on development’ and ‘Dominated plant effect’. |
| Weather/Locale | Adjustment of pollen viability per plant or timestep cohort. |
| Adjusted Population | Environment stressors include, but not limited to, those modeled by |
| Pollen viability | ‘Stress on development’ and ‘Dominated plant effect’. |
| Plant Population Start | Expressed as % of population. The number of plants beginning to |
| Shedding Pollen per | shed pollen within the population of male plants. |
| Time Step | |
| Plant Pollen Shedding | Expressed as Time Step Pollen Shed Per Plant. |
| Profile per Plant | |
| Percentage of Plants | The ratio of plants beginning to shed pollen to the total number of |
| Shedding Pollen | plants in the male plant population. |
| Daily Pollen Shed Per | Daily distribution of pollen shed per plant with variation in intensity |
| Plant | determined by total pollen/plant and pollen shed duration. |
| Number of Plants | The number of plants included in each time step cohort based on |
| | Plant population density and Daily Percentage of Plants to Start |
| | Shedding. |
| Temporal Dynamic of | The temporal profile of daily pollen shed density (grains/cm²) for all |
| Pollen Shed Density | time steps from 0 to 100% of the population shedding pollen |
| | generated from the daily pollen shed density for all pollen shedding |
| | cohorts. This relates to the development of a crop based on the |
| | calendar date in which it is planted. For example, a crop planted in |
| | May might shed pollen at a different rate than crops planted in June. |
| | Moreover, temporally-temporalbased plantings may serve as means |
| | to isolate fields or groups of plantsisolations as it relates to pollen |
| | shed. These values are used to calculate daily values for Percent of |
| | Stigmas Pollinated, converting receptive stigmas to seeds or fruits. |

TABLE 11
The outputs of the Plant Population Shedding Pollen Module.

| | |
|---|---|
| Cohorts Pollen | The cumulative intensity of pollen shed for all plants |
| Shedding | within the cohort. Calculation continues across time |
| | intervals until shed duration is reached. Daily |
| | summation of pollen shed intensity for all cohorts |
| | with actively shedding plants. This value is used to |
| | calculate Percent of Stigma Pollinated, which converts |
| | receptive stigmas to seeds or fruits. |
| Cohorts by | Levels of cohorts representing pollen shed over time. |
| Time Step | |

All of the inputs and calculations in the above modules lead to the Pollination Simulation Modules, Intentional Application Simulation Module, and the Calendar Module. The Pollination Simulation Module uses the input data and resulting calculations above to transform the data into a harvest of the seed, grain, or fruit of interest. More specifically, the Pollination Simulation Module predicts how many exserted stigmas are pollinated at each time step by natural pollination. The calculation is completed by determining whether pollen density at each time step limits pollination of all receptive stigmas available for pollination at that time step. Stigmas exposed to pollen of sufficient density are considered pollinated and removed from the available cohorts of remaining stigmas for subsequent time steps. Outputs of the Pollination Simulation Module for each time step include totals of all relevant cohorts for number of pollinations (equivalent to seed, grain, or fruit formed), cumulative pollination with time, and remaining unpollinated and receptive stigmas.

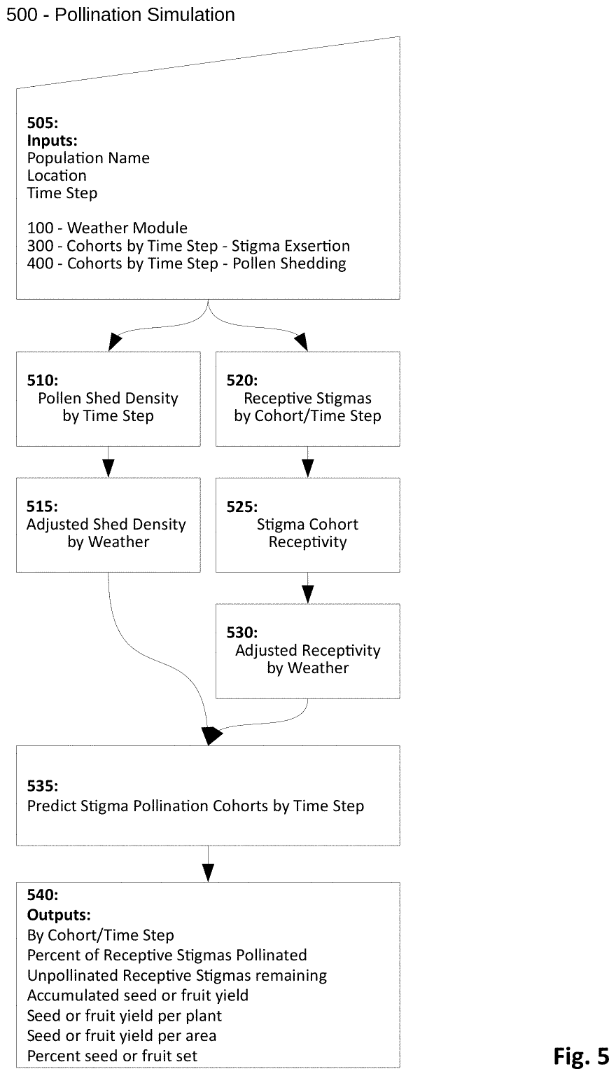

Referring to FIG. 5, an example of an embodiment of a Pollination Simulation Module is shown. Inputs include the time step, cohort name, location, and output from the Weather Module 100, Plant Population Stigma Exsertion Module 300, and the Plant Population Shedding Pollen Module 400. Outputs are shown in blocks 510, 515, 520, 525, 530, 535, and 540. As noted in block 540, output is calculated by cohort for each time step. The results may then be added for the final simulated results. Inputs, Calculations, and Outputs are described in more detail in tables 12, 13, and 14, respectively.

TABLE 12
Definitions of Inputs for Pollination Simulation Module.

| | |
|---|---|
| Time Step | The incremental change in time for which the method |
| | simulates pollination activities. Example: one day. |
| Cohort Name | The name given to a particular population of plants |
| | by a user. |
| Location | The location of the population of plants. In preferred |
| | embodiments, location may be inputted in latitude/ |
| | longitude inputs or GPS coordinate input. |
| Weather Module | One or more of the Weather Module outputs described |
| Data (100) | herein. |
| Cohorts by | Levels of cohorts representing stigma exsertion over |
| Time Step - | time. |
| Stigma | |
| Exsertion (300) | |
| Cohorts by | Levels of cohorts representing pollen shed over time. |
| Time Step - | |
| Pollen | |
| Shedding (400) | |

TABLE 13
Definitions of Calculations for Pollination Simulation Module.

| | |
|---|---|
| Pollen Shed | The actual density of pollen shedding from all |
| Density by | cohort sets per time step provided by the pollen |
| Time Step 510 | density calculations in the Population Adjustment |
| | Ratio Module described above. |
| Adjusted | Decrease calculated pollen density due to adverse |
| Pollen Shed | weather conditions such as rain, dew, wind, etc. |
| Density by | |
| Weather 515 | |
| Receptive | The actual number of receptive stigmas per area |
| Stigmas by | available for pollination in all cohort sets per |
| Cohort/Time | time step from population stigma exsertion |
| step | calculations. This is calculated in the Population |
| | Adjustment Ratio Module described above. |
| Stigma Cohort | Decrease % chance of stigma pollination due to age |
| Receptivity | and degrading efficacy. |
| Adjusted | Decrease % chance of stigma pollination due to adverse |
| Receptivity | weather conditions such as rain, dew, wind, etc. |
| by Weather | |
| Predict Stigma | Calculate number of receptive stigma per timestep |
| Pollination | cohort that are pollinated by current timestep of |
| Cohorts by | pollen density/availability. Create new cohorts of |
| Time Step | pollinated stigma per available cohorts/timesteps, |
| | making them unavailable for future timestep |
| | pollination calculations |

TABLE 14
Definitions of Outputs for Pollination Simulation Module.

| | |
|---|---|
| Percent of | A calculation to predict the percentage of exserted |
| Receptive | stigmas being pollinated per area based on the density |
| Stigmas | of pollen available. The fraction of unpollinated |
| Pollinated | receptive stigmas converted to seeds or fruits increases |
| | as a logistic function of pollen shed density up to a |
| | saturating density. The relationship is species specific |
| | reflecting variation in pollen-stigma interactions, |
| | pollen viability and vigor, and stigma receptivity. |
| Unpollinated | This calculation adjusts receptive stigmas per cohort |
| Receptive | each day by subtracting pollinated stigmas and |
| Stigmas | unpollinated stigmas that have exceeded the duration |
| Remaining | of stigma receptivity. Unpollinated stigmas remaining |
| | in the cohort are added to the next day's total for |
| | all cohorts exserting stigmas. |
| Accumulated | This calculation sums seed or fruit numbers predicted |
| Seed or Fruit | for all time intervals for which pollinations occurred. |
| Yield | |
| Seed or Fruit | Calculated ratio of seeds or fruit set per plant. |
| Yield per | |
| Plant | |
| Seed or Fruit | Calculated ratio of seeds or fruit set per area. |
| Yield per | |
| Area | |
| Percent Seed | Calculated ratio of seeds or fruit set per total number |
| or Fruit | of florets with exserted stigmas available for |
| Set | pollination. |

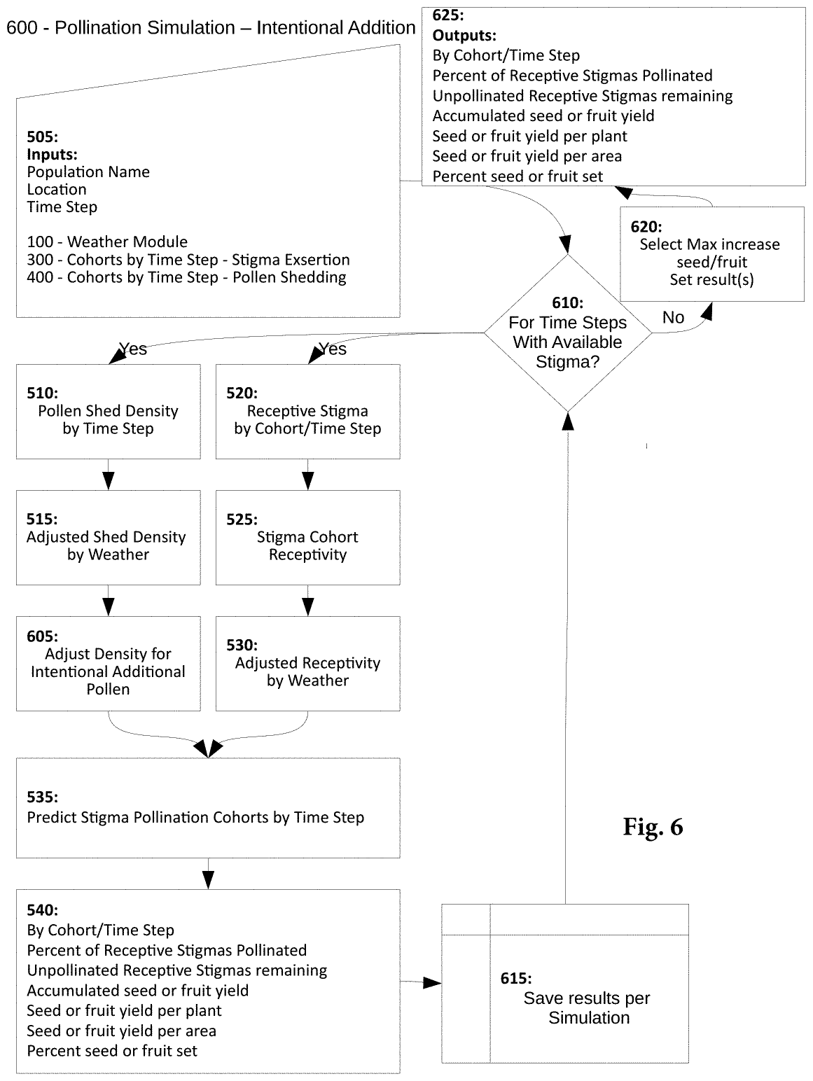

Referring to FIG. 6, the illustrated embodiment also includes an Intentional Pollination Simulation Module. Accordingly, in preferred embodiments, the Intentional Pollen Application Simulation Module provides the best day(s) on which to intentionally apply pollen to a population of plants. In some embodiments, this will be the only pollen that is applied population of plants. In other embodiments, the intentional pollination will provide supplemental pollen to plants that are also pollinated via natural pollination. The intentionally applied pollen may be fresh or preserved. The Intentional Pollen Application Simulation Module generates the best time step(s) for intentional pollen application by augmenting the pollen density cohorts. This is accomplished by interrogating each time step of the pollination simulation for receptive (sometimes called exposed) stigmas that remain unpollinated. In preferred embodiments, the Intentional Pollen Application Simulation Module includes a comparison of seed set in response to a saturating dose of pollen (defined as sufficient to ensure 97% seed set of exposed stigmas) with the seed set without intentional pollen application at leach time step to generate the best day(s) for pollen intentional pollen application. Accordingly, the system and method calculates the best time step for intentional pollen application based on the dynamic interactions of the female population, male population (if any), stigma exsertion rate, pollen shed per plant (if any), and duration of stigma receptivity, as well as current or expected weather conditions, which will be discussed in further detail below. Successful pollination takes into account weather conditions such as, but not limited to, humidity, vapor pressure deficit, temperature, water stress, wind speed, and precipitation, which can impact the success for seed and fruit formation. The complexity of these interactions renders this system of calculations and resulting prediction for ideal timing of pollen application neither obvious nor intuitive.

The Intentional Pollination Simulation Module is largely similar to the Pollination Simulation Module; however, it includes the addition of intentionally applied pollen in the simulation. Indeed, as noted in FIG. 6, blocks 505, 510, 515, 520, 525, 530, 535, and 540 are identical to the Pollination Simulation Module. Added, however, are blocks 605, 610, 615, 620, and 625. Referring first to block 605, this module adds the intentional pollen to pollen that is available for pollination. Referring to block 540, several output results are simulated by cohort for each time step. These results may be added to result in the total values for various time steps. Accordingly, output including percent of stigmas pollinated, unpollinated receptive stigmas remaining, accumulated seed or fruit yield, seed or fruit yield per plant, seed or fruit yield per area, and percent seed or fruit set are calculated for each time step with the availability of intentionally applied pollen. Referring to block 610, the simulation is carried out for each time step with available stigmas, and the results are saved 615. The simulation generates one or more time steps wherein harvest of the resulting seed, grain, and/or fruit are improved. Moreover, the simulation can rank the improvement. The simulated harvest of the resulting seed, grain, and/or fruit may be quantified and provided as output in block 625, which is identical to output 540 described in detail above, but with the availability of intentional pollen.

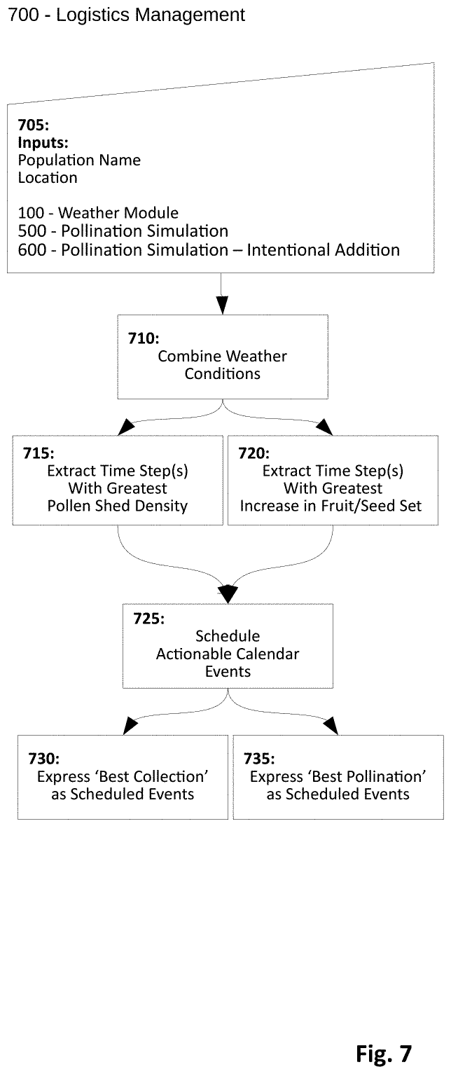

Referring to FIG. 7, embodiments of the method may include a Logistics Management Module 700, sometimes also referred to as a calendar module. Methods of the present invention are typically applied to several growing environments at the same time. For example, a grower could enter input and run the simulation(s) for a plurality of growing environments, such as a plurality of fields. The Logistics Management Module provides the user with a tool to simulate several growing environments at the same time and, thus, prioritize and manage the delivery of intentionally applied pollen across the plurality of growing environments (which will also be described below in association with the graphical user interfaces).

Inputs into the Logistics Management Module 700 incldue the Population Name, Location, and Weather Module 100 output, which have all been described in detail above. Further input includes output from the Pollination Simulation 500 and the Intentional Pollination Simulation 600 output. The method may include combining weather conditions 710 to extract the time step(s) with the greatest pollen shed density 715 and extract time steps(s) with the greatest increase in fruit, grain, and/or seed set 720. Those result in the scheduling of actionable calendar events. Specifically, the time step(s) with the greatest pollen shed density 715 result in the expression of ‘Best Collection’ as scheduled event(s) 730, while the time step(s) with the greatest increase in fruit, grain, and/or seed set 720 result in the expression of ‘Best Pollination’ as scheduled event(s) 735.

The output of the Intentional Application Simulation Module is consistent with the Pollination Simulation Module described above. The inputs and outputs of the Pollination Simulation Module are used, but they are combined with scenarios that involve providing intentional pollen application at each time step to determine which time step(s) having the intentional pollen application results in the most desirable seed, grain, and/or fruit results. In other words, the Intentional Application Simulation asks what would happen during each time step if pollination is not limited to natural pollination. This generates one or more best time steps to intentionally apply pollen to the population of plants.

The general logic of the calculations to determine the best day(s) to intentionally pollinate the crop converts the daily cohort of unpollinated, receptive florets into seed, grain, and/or fruit according to the daily density of pollen shed. The fraction of unpollinated, receptive florets converted to seeds or fruits increases as a logistic function of pollen shed density up to a saturating density. As an example, the saturating density of pollen shed to ensure 97% seed set of exposed receptive stigmas of maize is approximately 125 grains/cm².

As provided below in Table 15, the newly exposed female florets are calculated from the population dynamic of % female plants entering flowering×daily rate of stigma exposure per plant. Every plant is treated the same. In other words, in this specific calculation, there is no adjustment for different flowering rates on dominant or dominated plants. In other embodiments, the algorithms can be adjusted to account for different flowering rates on dominant or dominated plants. Total number of florets available for pollination each day (Cohort N) is the sum of newly exposed florets (increment in % of plants flowering×florets exposed on Dayⁿ) plus unpollinated, receptive florets from all prior daily cohorts (determined from rate and duration of stigma exsertion, duration of floret receptivity, and prior exposure to pollen density). Florets not pollinated on Dayⁿ are added to the next cohort exserted on Dayⁿ⁺¹, and so on. The duration of receptivity of unpollinated florets is a user selected variable. Thus, the actual number of florets available for pollination in each daily cohort=newly exposed florets (stigmas)+unpollinated florets from previous days—pollinated florets—senesced florets. Referring again to Table 15, in some embodiments, conditions that cause failure of some florets to reach anthesis can be incorporated in these calculations. In addition, loss of seeds or fruits due to abortion after pollination can be considered as well. The extent of post-pollination abortion or undeveloped florets might not be significant under irrigated and well managed conditions. But at high plant population densities or in stressful environments, these rates will can have a significant impact on final seed and fruit set and should be incorporated into the calculations to simulate the outcome of intentional pollination more accurately. The method, including the software version of the method, is designed to accommodate refinements to these calculations based on prior agronomic knowledge and impacts of weather on flowering dynamics and seed formation.

Daily density of pollen shed is calculated from the population dynamic of % shedding×daily pollen shed per plant. Each day a new cohort of plants begins to shed pollen (increment in % of plants beginning to shed pollen). The pollen added by each daily cohort follows the individual plant dynamic of shed×the number of plants engaged in pollen shed that day. The pollen shed summed from all cohorts is used to calculate the pollen shed density (grains/cm²) for the day. The effective (viable) pollen shed density then is adjusted for loss of viability prior to calculating seed or fruit set. The program can integrate the daily shed density for any number of male populations from which fresh pollen is collected for immediate application or storage for subsequent application, with calculations for each population managed independently. In addition, if desired, the diurnal pattern of pollen shed can be added as an additional, optional factor to be considered in the calculations. This factor is more significant for certain crops, such as corn, and is therefore not always a requirement.

Once the daily quantities of receptive stigmas/area and pollen shed density are established, the method converts receptive, unpollinated florets/area to seeds or grains or fruit per area in each relevant cohort and sums the values to determine the present seed or fruit set and daily addition of seeds/area or fruits/area.

The method provides the ability to plot the following developmental outputs:
1. Cumulative number of female florets/area
2. Daily pollen shed density
3. Cumulative number of seeds/area or fruits/area
4. Daily number of receptive florets not pollinated
5. Date of 50% female floral anthesis (stigmas exposed for pollination) for the population
6. Date of 50% male floral anthesis (beginning pollen shed) for the population

The method provides the ability to plot the following additional outputs:
1. Total florets/area
2. Total seeds/area or fruits/area
3. Percent seed set or percent fruit set
4. Average number of seeds or fruits per rachis
5. Average number of seeds or fruits per plant
6. Population profile of seeds or fruits per plant

The method provides the ability to analyze the following additional impacts on the effectiveness of intentional pollination and its impact on the crop output: (reference to FIGS.
1. Assessing the impact of varying the male plant population density on the crop output, which can help a grower make the best decisions pertaining to the layout of the growing environment (FIG. 2., blocks 205, 210, 215)
2. Assessing the impact of varying the female plant population density on the crop output, which can help a grower make the best decisions pertaining to the layout of the growing environment (FIG. 2., blocks 205, 210, 215)
3. Assessing the impact of altering the female and/or male planting configuration within the growing environment, which can help the grower make the best decisions pertaining to the layout of the growing environment (FIG. 2., blocks 205, 210, 215)
4. Assessing the impact of one or more intentional pollen applications for a female-only growing environment, allowing a grower to determine whether to reconsider adding a male to the growing environment, or to determine the optimal number of intentional applications of pollen for the greatest impact to yield or other crop characteristics (FIG. 6 blocks 540 vs 625)
5. Assessing the potential of one or more intentional pollination applications to decrease genetic impurity in the crop output (FIG. 6 blocks 540 vs 625)
6. Assessing the improvement in saleable seed from a given growing environment based on an expected percent seed set, which is a function of the intentional pollination timing (FIG. 6 blocks 540 vs 625)
7. Assessing the improvement in saleable grain from a given growing environment based on an expected percent grain set, which is a function of the intentional pollination timing (FIG. 6 blocks 540 vs 625)
8. Assessing the best sequence for application of said pollen across multiple fields, which is a function of flowering dynamics and timing of intentional pollination in each field (FIG. 6 blocks 540 vs 525; FIG. 7 blocks 725, 730, 735)
9. Assessing the optimal diurnal conditions for the intentional application of pollen after having established the best days for the application of said pollen. (FIG. blocks 105, 110; FIG. 6 blocks 540 vs 525; FIG. 7 blocks 705, 725, 730, 735)

The invention uses the developmental profiles of daily pollen shed density and the daily number of receptive florets not pollinated to determine the best opportunity to increase seed set, fruit set, and grain set or alter seed composition with an intentional application of pollen, whether primary or supplemental. For such prescriptive purposes, the method provides a saturating dose of intentionally applied pollen to the daily pollen shed dynamic, if any. This application converts the remaining unpollinated, receptive florets to seeds or fruits for each individual day of application. The method compares the potential increase in seed set or fruit set each day to the original daily values to determine the best day(s) to conduct the intentional pollination(s) based on the flowering dynamics of the male and female plant populations. The method also uses the developmental profile of daily pollen shed density to calculate the day(s) when maximum pollen shed will occur.

The method results can be presented in a calendar format as the range of ‘best pollen collection’ dates and ‘best intentional pollen application’ dates for each combination of male and female plants. There is no limit to the number of growing environments that can be compared simultaneously.

In at least one software embodiment of the invention, a sorting option enables the user to select subsets of growing environments for comparison. If agronomic values of growing degree units (GDUs) or accumulated heat units (AHUs) from planting to flowering are available for the plant species for which the method is being used, the method provides the initial prescriptions of ‘best pollen collection’ dates and ‘best intentional pollen application’ dates at planting based on long-term average weather or controlled environment conditions. Subsequent inputs on plant development, crop management, and weather can be used to refine the initial prescriptions.

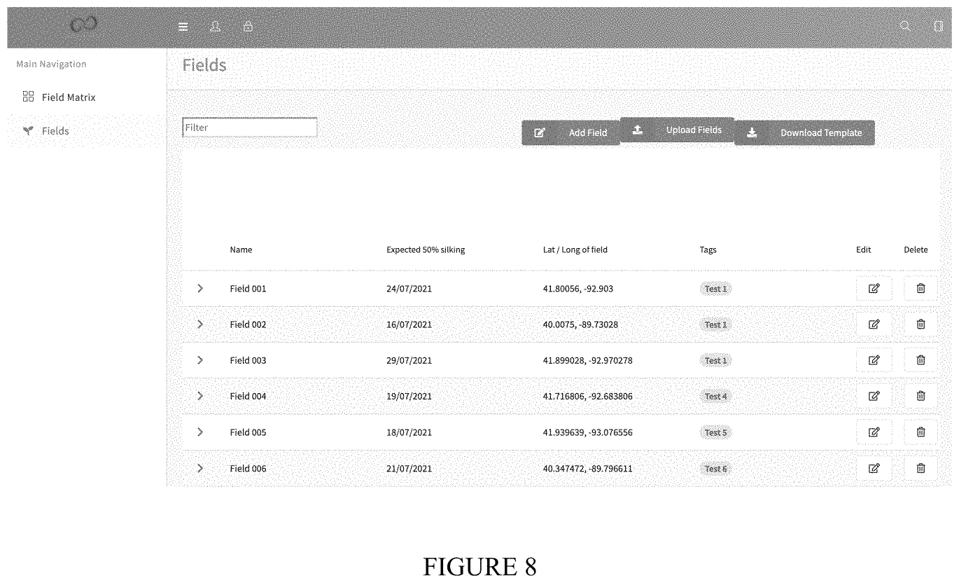

FIGS. 8-12 provide illustrations of graphical user interfaces of a computer-implemented embodiment of the present invention with respect to corn fields. FIG. 8 is an illustration of a field information page. It is anticipated that a user of the present invention will input information regarding a plurality of fields into the program. This screen lists the user's fields, along with some key information regarding same. For example, in this embodiment, the screen shows the name of the field, the date of expected 50% stigma exsertion (which in this case is 50% silking of a corn population), and the location of the field expressed in latitude and longitude. There are also buttons for a user to add a field, upload fields from a different program (such as Microsoft Excel), and an option to download a template for future uploading of fields.

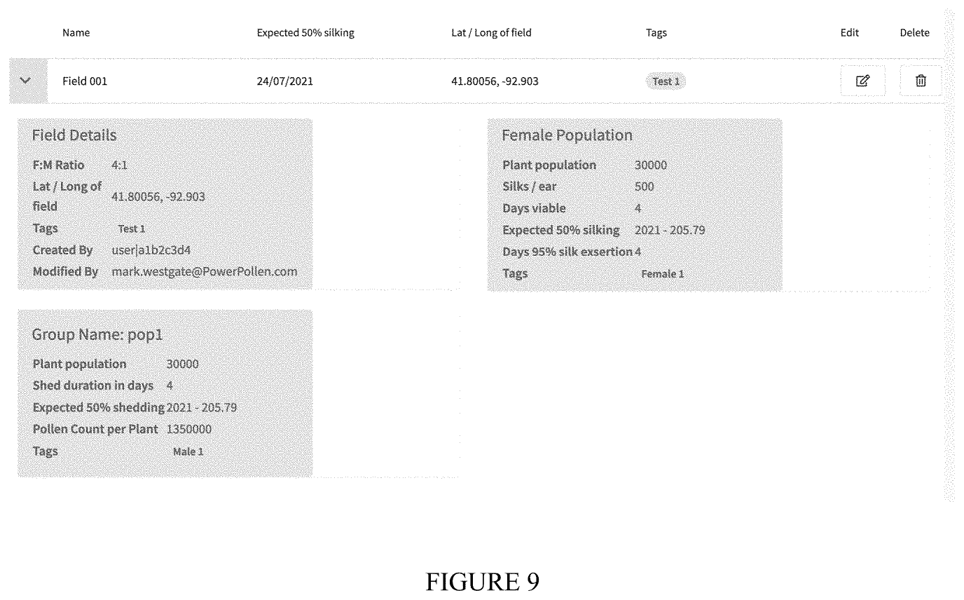

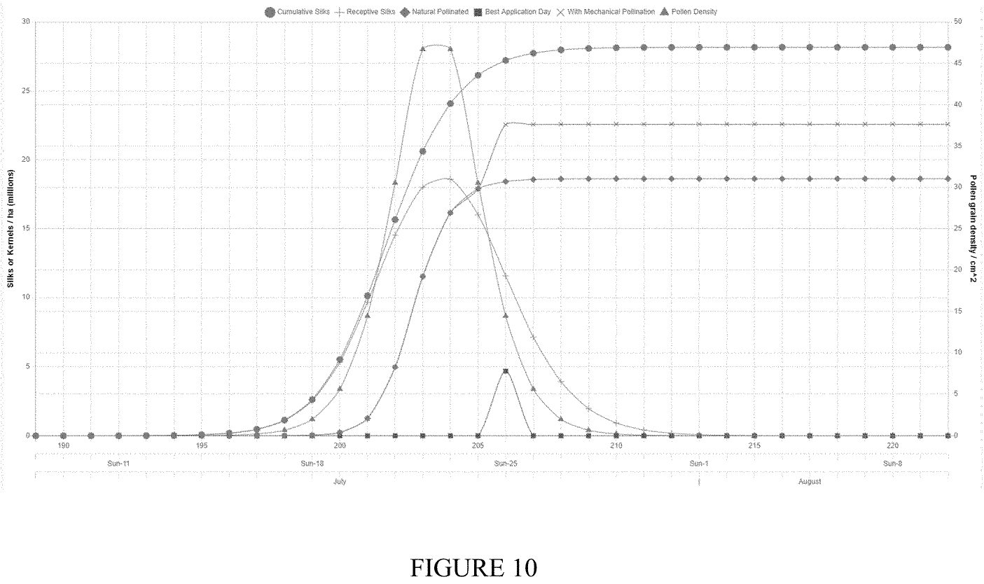

When a user clicks on a particular field, more information regarding that field is displayed. FIG. 9 illustrates such a screen. Shown on the screen are several details about the field, including inputs described above. These include the female to male plant ratio, location of the field, plant population, number of stigmas per plant (expressed as silks per ear), the number of days that the stigmas remain receptive to pollen, the date when 50% of the stigmas are expected to be exserted, the duration of pollen shed, the date when 50% of the males are expected to be shedding pollen, and the pollen count per plant. FIG. 10 also provides information regarding a particular field in graphical form. Namely, the illustrated graph shows the cumulative silks, the receptive silks, the naturally pollinated silks, the best application date, the best mechanical application day, and the pollen shed density over time, and more specifically, over days.

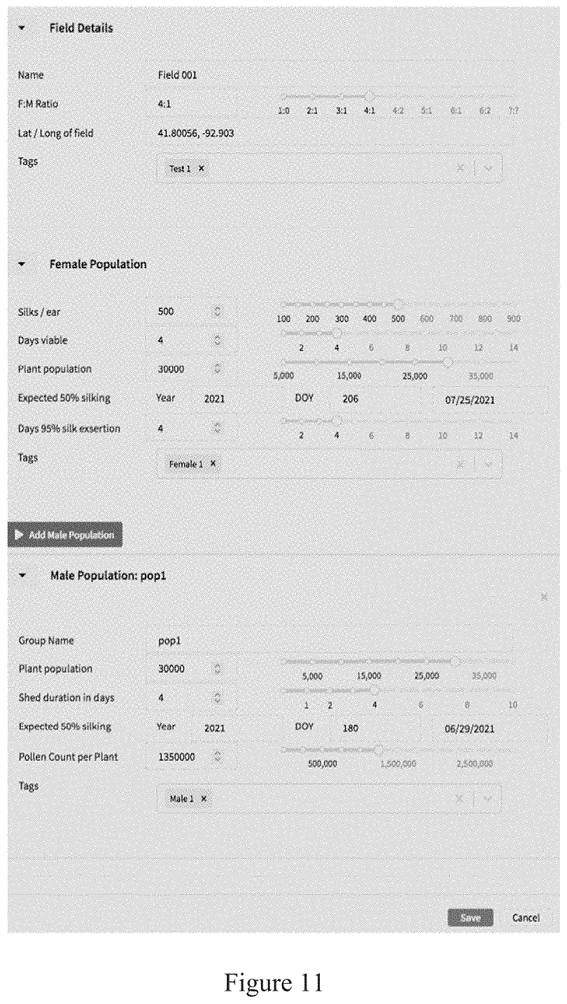

Referring to FIG. 11, when a user chooses to add a field, the screen illustrated in FIG. 3 appears. This screen provides a place for a user to enter several inputs required by the method. These include, the field name, female to male ratio, location expressed as latitude and longitude, stigmas per plant (expressed as silks per ear in this example), the number of days that females remain receptive to pollen, the number of plants in the population, the date when 50% of the plants are expected to have exserted stigmas, the day when 95% of the plants are expected to have exserted stigmas, the pollen shed duration, the day when 50% of the plants are expected to be shedding pollen, and the pollen count per plant.

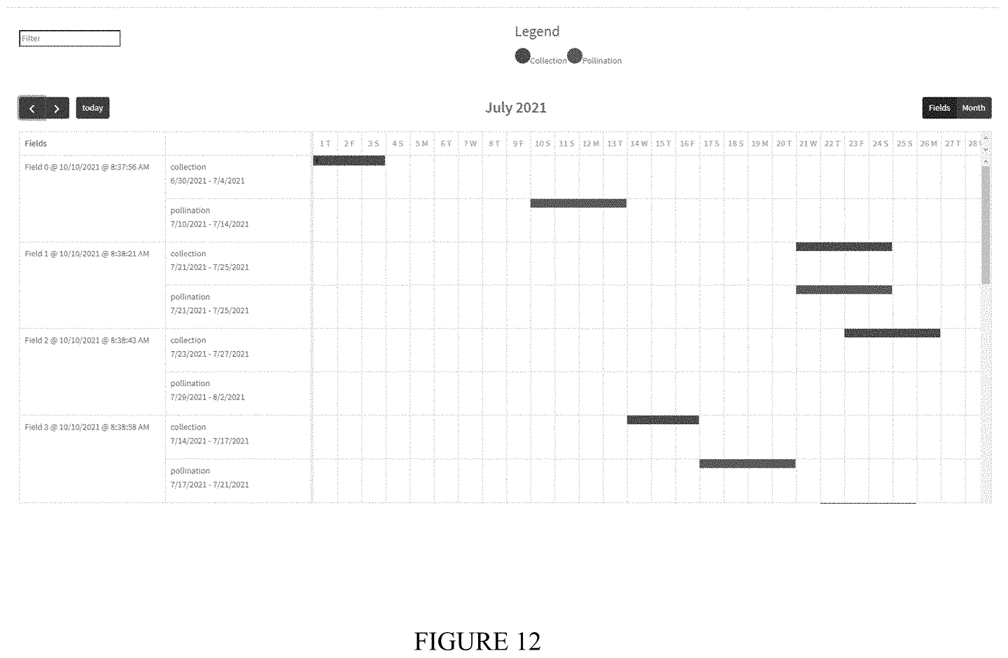

FIG. 12 provides a view of the calendar output of the present invention. It shows which days for each field result in the best pollen application, and in this embodiment also the best pollen collection. This allows a user to schedule and prioritize time in each field that will maximize desired output.

Furthermore, Table 15 below provides calculations related to all aspects of the method.

TABLE 15
Calculations related to an embodiment of a method of the present invention. Module and block numbers are
included as they are shown in the illustrated embodiment. However, a person of skill in the art will
recognize that the calculations may be used in one or more other illustrated modules or in a method using
different modules than those described herein without departing from the scope of the invention.

| | | | |
|---|---|---|---|
| | | Possible In-season | |
| Inputs | Assumptions | Adjustments | Notes |

Female Plants

| | | | |
|---|---|---|---|
| Planting density | Each plant can produce a seeds | Correction for | Variable |
| (plants per unit area) | or fruit | low germination | |
| (Block 215) | | or stand loss | |
| Separate inputs for | | | |
| each population of | | | |
| female plants | | | |
| (Blocks 205 and 305) | | | |
| Flowering dynamics | Population dynamic follows a | Adjust start date | Used to calculate |
| of the female plant | sigmoid function. | based on field | daily cohorts of plants |
| population. (Module | Basic equation: | scouting on progress | with new receptive |
| 300) | 100/(1 + exp(slope*(D₅₀ − D₀))). | of plant development | florets exposed for |
| Start date based on | | and population | pollination. Can |
| user provided growing | | uniformity assessed | incorporate weather |
| degree units (GDUs) | | prior to first | factors to alter slope |
| to flowering. | | flowers to reach | of population flowering |
| | | anthesis. | dynamic. |
| Maximum number | All florets are fertile when | Incorporate weather | Variable |
| of florets per rachis. | stigmas first exposed for | factors to alter | Adjusted to a value less |
| (Block 305) | pollination. | slope of stigma | than maximum floret number |
| | | exsertion dynamic. | based on prior observations |
| | | | of maximum seed set per |
| | | | rachis. |
| Rate/duration of | All florets eventually exsert | Incorporate weather | Used to calculate newly |
| stigma exsertion per | a receptive stigma | factors to alter | exposed receptive stigmas |
| rachis (Block 305) | Basic equation: | rate of stigma | within each daily cohort. |
| | A*(1 − exp (B*(Dₙ − Dᵢₙₜ)) | exsertion. | Combined with population |
| | A = floret number | | dynamic to calculate daily |
| | B = (2.9957/(D₉₅ − 0.7)). | | cohort of receptive florets |
| | Dₙ = day after first stigmas | | per area. |
| | exposed | | |
| | Dᵢₙₜ = X intercept | | |
| | D₉₅ = days to 95% stigmas | | |
| | exposed | | |
| Duration of female | Unpollinated florets eventually | Incorporate weather | Variable |
| floret receptivity | senesce if not pollinated | factors to alter | |
| (Block 305) | | duration of stigma | |
| | | receptivity | |
| Female:male | Determines ratio of female | | Variable |
| planting ratio (F:M | (stigma producing) to male | | Used to calculate Female |
| ratio) (Module 200) | (pollen producing) flowers per | | plants per area, and seeds |
| | area | | or fruits produced per area. |
| Daily percentage of | Female anthesis = exsertion of | | Variable |
| female plant | receptive stigmas | | |
| population reaching | | | |
| anthesis (optional) | | | |
| (Blocks 315, 320). | | | |
| Male Plants | | | |
| Planting density | Each plant produces flowers that | Correction for low | Variable |
| (pl/acre). | shed viable pollen | germination or stand | Used to calculate Male |
| (Module 200) | | loss | plants per field area |
| Separate inputs for | | | based on F:M ratio |
| each population of | | | |
| male plants | | | |
| (Modules 200 and 400) | | | |
| Pollen shed dynamics | Population dynamic follows a | Adjust start date and | Used to calculate a |
| of the population (% | sigmoid function. | slope based on scouting | daily cohort of plants |
| shedding). (Blocks | Basic equation: | data on progress of plant | shedding pollen. |
| 415, 420) | 100/(1 + exp(A*(D₅₀ − D₀))). | development and population | Can incorporate |
| Separate curves for | A = slope | uniformity assessed prior | weather factors to |
| inputs for different | D₅₀ = date of 50% shedding | to first male flowers to | alter slope of |
| populations of male | D₀ = shedding start date | reach anthesis. | flowering dynamic. |
| plants (Block 425) | | | |
| Plant pollen | All plants follow a quasi-normal | Incorporate stress/weather | Variable |
| shedding profile per | pollen shed distribution. | factors to alter duration | Used to calculate cohorts |
| plant. (Block 420) | All pollen shed is viable and | per plant, or daily shed | pollen shedding by time |
| | capable of fertilizing receptive | intensity. | step from pollen/plant |
| | female florets | Incorporate a temperature | and shed duration. |
| | Basic equation: | correction for pollen | |
| | f(x) = ((A/(B*(3.1416/ | viability or fertility. | |
| | 2)\hat{ }0.5))*EXP(−2*((x − C)/B)\hat{ }2)) | | |
| | A = pollen per plant | | |
| | B = variance | | |
| | C = peak | | |
| Stress on pollen | f(x) = B − (1 + P)\hat{ }(N − A) | Weather Module translates | Maximum value of pollen |
| viability (Block | B = maximum value, N = stress | historical, current, and | viability is species |
| 411) | level (arbitrary units), P & A | forecast weather data to | specific input |
| | species specific coefficients | stress level units 0-10. | |
| Conversion of male | Pollen shed density determines | | Calculated daily for |
| and female | the fraction of receptive female | | each cohort of |
| flowering dynamics | florets that set seed. | | receptive female |
| to daily seed set | Basic equation: | | flowers. |
| (Blocks 535, 540) | f(x) = 0, for x = 0 | | |
| | f(x) = y₀ + A/(1 + exp(−(x − x₀)/B)), | | |
| | for x > 0 | | |
| | x = daily pollen shed density | | |
| | (grains/cm\hat{ }2) | | |
| | A, B are constants specific to | | |
| | species | | |
| Stress on seed fruit | f(x) = B − (1 + P)\hat{ }(N − A) | Weather Module translates | Basal, mid, and apical |
| Abortion (Block | B = maximum value (100%), N = | historical, current, and | florets assigned to |
| 311) | stress level (arbitrary units), P & | forecast weather data to | time step cohorts based |
| | A species specific coefficients | stress level units 0-10. | on date of exsertion. |
| Stress on | f(x) = B − (1 + P)\hat{ }(N − A) | Weather Module translates | Basal, mid, and apical |
| development (Block | B = maximum value (100%), N = | historical, current, and | florets assigned to |
| 312) | stress level (arbitrary units), P & | forecast weather data to | time step cohorts based |
| | A species specific coefficients | stress level units 0-10. | on date of exsertion. |
| Dominated Plant | f(x) = B − (1 + P)\hat{ }(N − A) | Output decreases stigma | Calculation assumes first |
| Effect (Block 313) | B = maximum value (100%), N | exsertion rate by | plants in the population |
| | = cohort number for plants | indicated percentage | to exsert stigmas are |
| | beginning to exsert stigmas, P & | for dominated plants in | dominant plants. Those |
| | A species specific coefficients | cohorts beginning to | beginning to exsert stigmas |
| | | exsert stigmas later in | late are dominated plants. |
| | | the population. | |

Accordingly, in light of the above disclosure, the invention further provides a method of determining when to pollinate a crop plant, and optionally determining one or more time steps (including but not limited to a day), time points, and/or time periods on which intentional pollination can be optimized, for example to provide the greatest or greater output (such as harvest) of seed, grain, and/or fruit of interest. Such a method need not include a pollination step, and optionally excludes a pollination step, but rather is a method of determining when to intentionally pollinate a population or portion of a population of plants of interest. For example, such a method may be defined by one or more of the following numbered sub-paragraphs:
1. A method for determining one or more time steps (including but not limited to a day), time points, and/or time periods on which to optimize intentional pollination of a crop plant having one or more stigmas that are receptive to pollen and that produces at least one seed, grain, or fruit of interest, said method comprising, consisting essentially of, or consisting of:
   a. Ingesting, as input data, reproductive maturity data for a population of said crop plant, wherein said reproductive maturity data includes information sufficient to determine one or more days on which said crop plant will be receptive to pollen; and
   b. Modeling the input data in a plurality of data processing modules within a computing environment with at least one processor, the data processing modules configured to identify one or more time steps during which to intentionally pollinate said population of said crop, by:
      i. Generating the amount of receptive stigmas in the population during a plurality of time steps; and
      ii. Modeling the effect of intentionally applied pollen during each time step to transform the number of receptive stigmas during each time step into a modeled output of said seed, grain, or fruit of interest; and
      iii. Generating one or more time steps during which intentional pollination is modeled to provide a greater harvest of said seed, grain, or fruit of interest than other of said time steps.
2. The method of sub-paragraph 1 further comprising modeling the availability of pollen for natural pollination during each time step.
3. The method of sub-paragraphs 1 or 2 wherein said modeling the availability of pollen for natural pollination during each time step includes:
   a. Modeling the amount of available pollen during each time step, and/or
   b. Modeling the number of stigmas that are naturally pollinated during each time step.
4. The method of any of sub-paragraph 1-3 wherein said time step is one day.
5. The method of any of sub-paragraphs 1-4 wherein said crop plant is corn.
6. The method of any of sub-paragraphs 1-5 wherein the pollen considered for the purposes of the modelling during the intentional pollination step is selected from the group consisting of fresh pollen, preserved pollen, and combinations thereof.
7. The method of sub-paragraph 6 wherein said pollen is preserved pollen.
8. The method of any of sub-paragraph 1-7 wherein said reproductive maturity data sufficient to determine one or more days on which said crop plant will be receptive to pollen includes one or more of:
   a. The amount of time needed between planting said crop and said crop beginning to exsert stigmas that are receptive to pollen;
   b. The amount of heat units that are needed for said crop to exsert stigmas that are receptive to pollen;
   c. The number of stigmas per plant;
   d. The rate at which said crop exserts stigmas that are receptive to pollen; and/or
   e. The number of time steps during which said crop's exserted stigmas remain receptive to pollen.
9. The method of any of sub-paragraphs 1-8 wherein modeling the availability of pollen for natural pollination during each time step includes ingesting data related to pollen shed, wherein said data related to pollen shed includes one or more of:
   a. The amount of time needed between planting one or more plants that will shed pollen and said one or more plants that will shed pollen beginning to shed said pollen;
   b. The amount of heat units that are needed between planting one or more plants that will shed pollen and said one or more plants that will shed pollen beginning to shed said pollen;
   c. The amount of pollen shed from each plant that will shed pollen;
   d. The rate at which said plant that will shed pollen sheds pollen; and/or
   e. The number of time steps during which said plant that will shed pollen sheds pollen.
10. The method of any of sub-paragraphs 1-9 wherein said method is applied to crop plants having one or more stigmas that are receptive to pollen in a plurality of growing environments and said method generates one or more time steps for each growing environment during which intentional pollination is modeled to provide a greater harvest of said seed, grain, or fruit of interest than others of said time steps.
11. The method of sub-paragraph 10 wherein said plurality of growing environments are a plurality of fields in different locations.
12. The method of sub-paragraph 10 or 11 further comprising generating a calendar of said time steps for each growing environment during which intentional pollination is modeled to provide a greater harvest of said seed, grain, or fruit of interest than others of said time steps.
13. The method of any of sub-paragraphs 1-12 wherein said pollination is cross-pollination.
14. The method of any of sub-paragraphs 1-12 wherein the input data further comprises weather data that includes one or more of:
   a. Historical weather data;
   b. Current day weather data; and
   c. Forecasted weather data.
15. The method of any of sub-paragraphs 1-14 and 16, wherein the practice of the method increases the value of the harvest.
16. A method for determining one or more time steps (including but not limited to a day), time points, and/or time periods on which to optimize intentional pollination of a crop plant having one or more stigmas that are receptive to pollen and that produces at least one seed, grain, or fruit of interest, said method comprising, consisting essentially of, or consisting of:
   a. Ingesting, as input data, reproductive maturity data for a population of said crop plant, wherein said reproductive maturity data includes information sufficient to determine one or more days on which said crop plant will be receptive to pollen;
   b. Modeling the input data to identify one or more time steps during which to intentionally pollinate said population of said crop, by:
      i. Generating the amount of receptive stigmas in the population during a plurality of time steps;
      ii. Modeling the effect of intentionally applied pollen during each time step to transform the number of receptive stigmas during each time step into a modeled output of said seed, grain, or fruit of interest; and
      iii. Generating one or more time steps during which intentional pollination is modeled to provide a greater harvest of said seed, grain, or fruit of interest than other of said time steps.
17. The method of any of sub-paragraphs 1-15 may be a computer-implemented method.
18. The method of any of sub-paragraphs 1-16 may be a method for pollinating a crop plant and further include intentionally pollinating the population of the crop plant during the one or more time steps during which intentional pollination is modeled to provide a greater harvest of said seed, grain, or fruit of interest than other of said time steps.

Also disclosed are methods of pollination, methods of grain production, methods of seed production, and/or methods of fruit production, including said methods of the present invention as described elsewhere in the present application, wherein downstream uses of the grain, seed, and/or fruit exclude grain, seed, and/or fruit used for the purposes of plant breeding and/or involves the destruction of the grain, seed, and/or fruit. Uses of such grain, seed, and/or fruit may include, but are not limited to, animal feed, fuel and uses in the production thereof (including but not limited to ethanol) for example as a feedstuff for fermentation in production of said fuel, food for human consumption, and industrial uses excluding plant breeding. Moreover, such methods may exclude essentially biological processes for the production of plants.

Moreover, also disclosed is a population of crop plants, characterized in that the plants in the population have been pollinated according to a method of the present invention as described herein, for example by the following method: (1) Ingesting, as input data, reproductive maturity data for a population of said crop plant, wherein said reproductive maturity data includes information sufficient to determine one or more days on which said crop plant will be receptive to pollen; (2) Modeling the input data to identify one or more time steps during which to intentionally pollinate said population of said crop, by: (i) Generating the amount of receptive stigmas in the population during a plurality of time steps; (ii) Modeling the effect of intentionally applied pollen during each time step to transform the number of receptive stigmas during each time step into a modeled output of said seed, grain, or fruit of interest; and (iii) Generating one or more time steps during which intentional pollination is modeled to provide a greater harvest of said seed, grain, or fruit of interest than other of said time steps; and (3) Intentionally pollinating said population of said crop plant during at least one of said time steps during which intentional pollination is modeled to provide a greater harvest of said seed, grain, or fruit of interest than other of said time steps.

Also disclosed is a population of crop plants, characterized in that the plants in the population have been pollinated according to a method of the present invention as described herein, for example by the following method: (1) Ingesting, as input data, reproductive maturity data for a population of said crop plant, wherein said reproductive maturity data includes information sufficient to determine one or more days on which said crop plant will be receptive to pollen; (2) Modeling the input data in a plurality of data processing modules within a computing environment with at least one processor, the data processing modules configured to identify one or more time steps during which to intentionally pollinate said population of said crop, by: (i) Generating the amount of receptive stigmas in the population during a plurality of time steps; (ii) Modeling the effect of intentionally applied pollen during each time step to transform the number of receptive stigmas during each time step into a modeled output of said seed, grain, or fruit of interest; and (iii) Generating one or more time steps during which intentional pollination is modeled to provide a greater harvest of said seed, grain, or fruit of interest than other of said time steps; and (3) Intentionally pollinating said population of said crop plant during at least one of said time steps during which intentional pollination is modeled to provide a greater harvest of said seed, grain, or fruit of interest than other of said time steps.

Moreover, also provided is a method for simulating pollination of a crop plant having one or more stigmas that are receptive to pollen and that produces at least one seed, grain, or fruit of interest, wherein the method comprises (1) Ingesting, as input data, reproductive maturity data for a population of said crop plant, wherein said reproductive maturity data includes information sufficient to determine one or more days on which said crop plant will be receptive to pollen; and (2) Modeling the input data to identify one or more time steps during which to intentionally pollinate said population of said crop, by: (i) Generating the amount of receptive stigmas in the population during a plurality of time steps; (ii) Modeling the effect of intentionally applied pollen during each time step to transform the number of receptive stigmas during each time step into a modeled output of said seed, grain, or fruit of interest; and (iii) Generating one or more time steps during which intentional pollination is modeled to provide a greater harvest of said seed, grain, or fruit of interest than other of said time steps. Such a method may be completed in silico, although it need not be.

Further disclosed is a method for simulating pollination of a crop plant having one or more stigmas that are receptive to pollen and that produces at least one seed, grain, or fruit of interest, wherein the method comprises (1) Ingesting, as input data, reproductive maturity data for a population of said crop plant, wherein said reproductive maturity data includes information sufficient to determine one or more days on which said crop plant will be receptive to pollen; (2) Modeling the input data in a plurality of data processing modules within a computing environment with at least one processor, the data processing modules configured to identify one or more time steps during which to intentionally pollinate said population of said crop, by: (i) Generating the amount of receptive stigmas in the population during a plurality of time steps; (ii) Modeling the effect of intentionally applied pollen during each time step to transform the number of receptive stigmas during each time step into a modeled output of said seed, grain, or fruit of interest; and (iii) Generating one or more time steps during which intentional pollination is modeled to provide a greater harvest of said seed, grain, or fruit of interest than other of said time steps; and (3) Intentionally pollinating said population of said crop plant during at least one of said time steps during which intentional pollination is modeled to provide a greater harvest of said seed, grain, or fruit of interest than other of said time steps. Such a method may be completed in silico, although it need not be.

Referring to paragraphs 0093, 0094, 0095, 0096, 0097, and/or 0098, the minimum number of plants in such a population may be any number and will be dependent on the type of crop. Moreover, the percentage of plants of the population that were pollinated by the method on the same day may include 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 95% or more, 97% or more, 98% or more, 99% or more, or 100%.

Referring to paragraphs 0093, 0094, 0095, 0096, 0097, 0098, and/or 0099, the pollen used for intentional pollination may be preserved pollen. The percentage of plants in the population pollinated by intentional application of preserved pollen may be 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 95% or more, 97% or more, 98% or more, 99% or more, or 100%.

Referring to paragraphs 0093, 0094, 0095, 0096, 0097, 0098, 0099, and/or 0100 the pollination may occur in a growing environment including, but not limited to, a field, shade house, glass house, greenhouse, hoop house, growth chamber, vertical farming facility, hydroponic facility, and/or aeroponic facility.

Also disclosed are a computer program, computer program product, and computing apparatus configured to carry out all or a portion of the described methods that relate to purely cognitive tasks, pertaining to the input, processing and output of data. In particular, although not exclusively, computer programs, products and apparatus may be configured to perform the methods disclosed in paragraphs 0093, 0094, 0095, 0096, 0097, 0098, 0099, 0100, and/or 0101, including any optional features of those methods described elsewhere herein.

Although various representative embodiments of this invention have been described above with a certain degree of particularity, those skilled in the art could make numerous alterations to the disclosed embodiments without departing from the spirit or scope of the inventive subject matter set forth in the specification and claims. In some instances, in methodologies directly or indirectly set forth herein, various steps and operations are described in one possible order of operation, but those skilled in the art will recognize that steps and operations may be rearranged, replaced, or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in the appended claims.

Although the present invention has been described with reference to the embodiments outlined above, various alternatives, modifications, variations, improvements and/or substantial equivalents, whether known or that are or may be presently foreseen, may become apparent to those having at least ordinary skill in the art. Listing the steps of a method in a certain order does not constitute any limitation on the order of the steps of the method. Accordingly, the embodiments of the invention set forth above are intended to be illustrative, not limiting. Persons skilled in the art will recognize that changes may be made in form and detail without departing from the spirit and scope of the invention. Therefore, the invention is intended to embrace all known or earlier developed alternatives, modifications, variations, improvements, and/or substantial equivalents.

1. A method for pollinating a crop plant having one or more stigmas that are receptive to pollen and that produces at least one seed, grain, or fruit of interest, said method comprising:
a. Ingesting, as input data, reproductive maturity data for a population of said crop plant, wherein said reproductive maturity data includes information sufficient to determine one or more days on which said crop plant will be receptive to pollen;
b. Modeling the input data in a plurality of data processing modules within a computing environment with at least one processor, the data processing modules configured to identify one or more time steps during which to intentionally pollinate said population of said crop, by:
i. Generating the amount of receptive stigmas in the population during a plurality of time steps;
ii. Modeling the effect of intentionally applied pollen during each time step to transform the number of receptive stigmas during each time step into a modeled output of said seed, grain, or fruit of interest; and
iii. Generating one or more time steps during which intentional pollination is modeled to provide a greater harvest of said seed, grain, or fruit of interest than other of said time steps; and
c. intentionally pollinating said population of said crop plant during at least one of said time steps during which intentional pollination is modeled to provide a greater harvest of said seed, grain, or fruit of interest than other of said time steps.

2. The method of claim 1 further comprising modeling the availability of pollen for natural pollination during each time step.

3. The method of claim 2 wherein said modeling the availability of pollen for natural pollination during each time step includes:
a. Modeling the amount of available pollen during each time step.
b. Modeling the number of stigmas that are naturally pollinated during each time step.

4. The method of claim 3 wherein said time step is one day.

5. The method of claim 4 wherein said crop plant is corn.

6. The method of claim 5 wherein pollen applied during the intentional pollination step is selected from the group consisting of fresh pollen, preserved pollen, and combinations thereof.

7. The method of claim 6 wherein said pollen is preserved pollen.

8. The method of claim 5 wherein said reproductive maturity data sufficient to determine one or more days on which said crop plant will be receptive to pollen includes one or more of:
a. The amount of time needed between planting said crop and said crop beginning to exsert stigmas that are receptive to pollen;
b. The amount of heat units that are needed for said crop to exsert stigmas that are receptive to pollen;
c. The number of stigmas per plant;
d. The rate at which said crop exserts stigmas that are receptive to pollen;
e. The number of time steps during which said crop's exserted stigmas remain receptive to pollen.

9. The method of claim 8 wherein modeling the availability of pollen for natural pollination during each time step includes ingesting data related to pollen shed, wherein said data related to pollen shed includes one or more of:
a. The amount of time needed between planting one or more plants that will shed pollen and said one or more plants that will shed pollen beginning to shed said pollen;
b. The amount of heat units that are needed between planting one or more plants that will shed pollen and said one or more plants that will shed pollen beginning to shed said pollen;
c. The amount of pollen shed from each plant that will shed pollen;
d. The rate at which said plant that will shed pollen sheds pollen;
e. The number of time steps during which said plant that will shed pollen sheds pollen.

10. The method of claim 5 wherein said method is applied to crop plants having one or more stigmas that are receptive to pollen in a plurality of growing environments and said method generates one or more time steps for each growing environment during which intentional pollination is modeled to provide a greater harvest of said seed, grain, or fruit of interest than others of said time steps.

11. The method of claim 10 wherein said plurality of growing environments are a plurality of fields in different locations.

12. The method of claim 10 further comprising generating a calendar of said time steps for each growing environment during which intentional pollination is modeled to provide a greater harvest of said seed, grain, or fruit of interest than others of said time steps.

13. The method of claim 1 wherein said pollination is cross-pollination.

14. The method of claim 9 wherein the input data further comprises weather data that includes one or more of:
a. Historical weather data;
b. Current day weather data; and
c. Forecasted weather data.

15. The method of claim 1, wherein the practice of the method increases the value of the harvest.

16. A method for pollinating a crop plant having one or more stigmas that are receptive to pollen and that produces at least one seed, grain, or fruit of interest, said method comprising:
a. Ingesting, as input data, reproductive maturity data for a population of said crop plant, wherein said reproductive maturity data includes information sufficient to determine one or more days on which said crop plant will be receptive to pollen;
b. Modeling the input data to identify one or more time steps during which to intentionally pollinate said population of said crop, by:
i. Generating the amount of receptive stigmas in the population during a plurality of time steps;
ii. Modeling the effect of intentionally applied pollen during each time step to transform the number of receptive stigmas during each time step into a modeled output of said seed, grain, or fruit of interest; and
iii. Generating one or more time steps during which intentional pollination is modeled to provide a greater harvest of said seed, grain, or fruit of interest than other of said time steps; and
c. Intentionally pollinating said population of said crop plant during at least one of said time steps during which intentional pollination is modeled to provide a greater harvest of said seed, grain, or fruit of interest than other of said time steps.
